(19) United States
(12) Patent Application Publication
(10) Pub. No.: US 2022/0280513 A1
(43) Pub. Date: Sep. 8, 2022

(54) ENHANCING THE ANTIVIRAL EFFICACY OF RNA VIRUS INHIBITION BY COMBINATION WITH MODULATORS OF PYRIMIDINE METABOLISM

(71) Applicant: The Board of Trustees of the Leland Stanford Junior University, Stanford, CA (US)

(72) Inventors: Qi Liu, Milbrae, CA (US); Chaitan Khosla, Stanford, CA (US); Amita Gupta, Stanford, CA (US)

(21) Appl. No.: 17/625,592

(22) PCT Filed: Jul. 14, 2020
(86) PCT No.: PCT/US2020/041986

§ 371 Date: Jan. 7, 2022

Related U.S. Application Data
(60) Provisional application No. 62/875,790, filed on Jul. 18, 2019.

(51) Int. Cl. A61K 31/513; A61K 31/7064; A61P 31/14; A61K 9/00

(52) U.S. Cl. CPC A61K 31/513; A61K 31/7064; A61P 31/14; A61K 9/0019

(57) ABSTRACT

Compounds and methods are provided for the treatment of pathogenic virus infections. Compositions and methods are provided for inhibiting RNA viruses.

### CROSS REFERENCE

This application claims benefit of U.S. Provisional Patent Application No. 62/875,790 filed Jul. 18, 2019, which applications are incorporated herein by reference in their entirety.

### GOVERNMENT SUPPORT

This invention was made with Government support under contract 1U19 AI109662, awarded by the National Institutes of Health. The Government has certain rights in this invention.

### BACKGROUND

The rapid rise in the number of emerging pathogens in the world's population represents a serious global health problem and underscores the need to develop broad spectrum anti-infectives that target common components of large classes of pathogens. Targeting viral proteins has the inextricable challenge of rise of resistance. Safe and effective vaccines are not possible for many viral pathogens. New approaches are required to address the unmet medical need in this area.

An understanding of the molecular mechanisms of viral life cycles has led to the identification of viral proteins as targets for therapeutic intervention, however few effective and safe agents have emerged, and these face the challenge of high mutation rates that have confounded many conventional antiviral products.

As an alternative to targeting viral proteins, targeting a cellular protein may lead to antiviral compounds with a broader spectrum of activity and less opportunity for developing resistance. However, targeting the host may result in toxicity, especially if the protein or pathway used is crucial for cell survival. The present invention provides broad spectrum anti-infective agents for use in treating viral infections.

### SUMMARY OF THE DISCLOSURE

Compositions and methods are provided for inhibiting RNA viruses. Replication of an RNA virus is inhibited by contacting infected cells, or cells at risk of infection, with a combination of an analog of cyclopentenyl uracil (CPU), an inhibitor of pyrimidine salvage, and an inhibitor of de novo pyrimidine synthesis, including inhibitors of dihydroorotate dehydrogenase (DHODH). In some embodiments the inhibitors of mammalian pyrimidine metabolism are administered in combination with an inhibitor of RNA dependent RNA polymerase (RdRp). This combination therapy is shown herein to markedly increased the potency of RNA-dependent RNA polymerase (RdRp) inhibition.

A benefit of the present invention can be the dose of pyrimidine salvage inhibitor in combination with an inhibitor of de novo pyrimidine synthesis, including DHODH inhibitors, to improve the antiviral efficacy of the stand-alone treatment with de novo synthesis inhibitors. A benefit of the combination can be the use of lowered doses of the agents, e.g. the RdRp inhibitor relative to the dose required as a single agent, which may reduce side-effects and allow drugs that have undesirable toxicity at single agent dosing to be used clinically. A benefit of the present invention can also, or alternatively, be a decrease in the length of time required for treatment, relative to the length of time required for treatment as a single agent. A benefit of the present invention can also, or alternatively, be an enhanced response relative to the response observed after treatment with a single agent. A benefit of the present invention can also, or alternatively, be use of an agent against viruses for which the therapeutic index is low as a single agent, relative to the use for treatment as a single agent. In some embodiments the combination of active agents provides for decreased toxicity to the host. In some embodiments the dosage and ratio of agents is selected to achieve increased efficacy with reduced toxicity, particularly reduced toxicity relative to the administration of the RdRp inhibitor as a single agent. Indicia of toxicity may include, without limitation, leukopenia, and other effects on rapidly dividing cells.

In some embodiments, the CPU analog has a structure as shown in FIG. 3 or FIG. 4. In some embodiments the CPU analog has a structure selected from the following group:

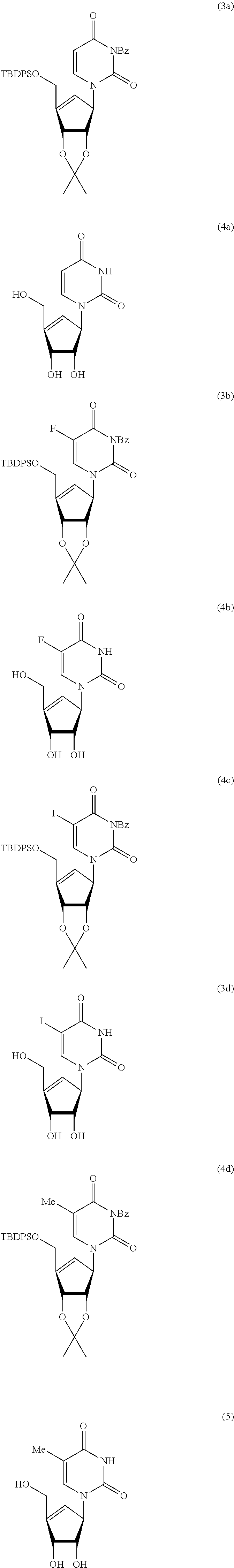

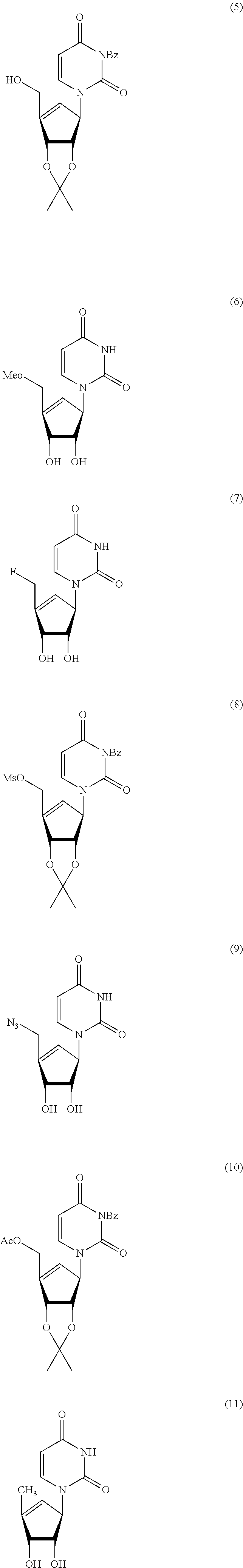

In some embodiments the CPU analog is one or both of ((3aS,4R,6aR)-4-(3-benzoyl-2,4-dioxo-3,4-dihydropyrimidin-1(2H)-yl)-2,2-dimethyl-3a,6a-dihydro-4H-cyclopenta[d][1,3]dioxol-6-yl)methyl methanesulfonate (structure 8 above), and 1-((1R,4R,5 S)-4,5-dihydroxy-3-(hydroxymethyl)cyclopent-2-en-1-yl)-5-fluoropyrimidine-2,4(1H,3H)-dione (structure 4b above).

In some embodiments a CPU analog has a structure selected from the following group:

\textcircled{B} has a structure selected from the following group:

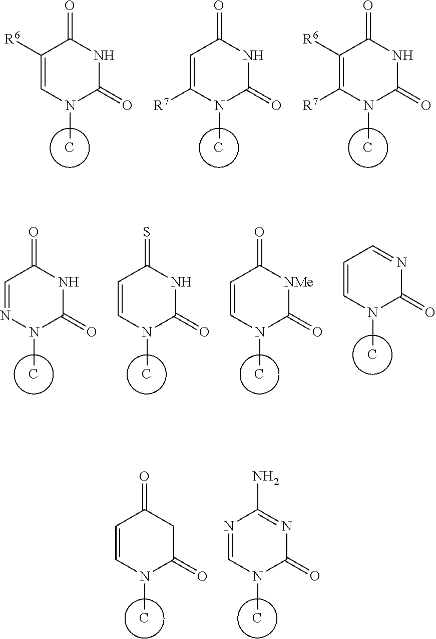

\textcircled{C} has a structure selected from the following group:

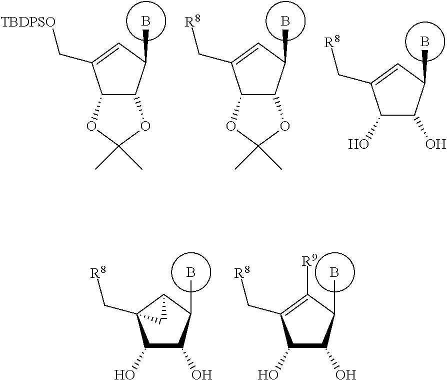

R⁶, R⁷, R⁸ and R⁹ is the same or different and is independently selected from the group consisting of halogen, haloalkyl, alkyl, akenyl, alkynyl, cycloalkyl, cycloalkenyl, —R¹⁰cycloalkyl, Ay, —NHR¹⁰Ay, Het, —NHHet, —NHR¹⁰Het, —OR²—OAy, —OHet, —R¹⁰OR², —NR²R³, —NR²Ay, —R¹⁰NR²R³, —R¹⁰NR²Ay, —R¹⁰C(O)R², —C(O)R², —CO₂R², —R¹⁰CO₂R², —C(O)NR²R³, —C(O)Ay, —C(O)NR²Ay, —C(O)Het, —C(O)NHR¹⁰Het, —R¹⁰C(O)NR²R³, —C(S)NR²R³, —R¹⁰C(S)NR²R³, —R¹⁰NHC(NH)NR²R³, —C(NH)NR²R³, —R¹⁰C(NH)NR²R³, —S(O)₂NR²R³, —S(O)₂NR²Ay, —R¹⁰SO₂NHCOR², —R¹⁰SO₂NR²R³, —R¹⁰SO₂R², —S(O)ₘR², —S(O)Ay, cyano, nitro, or azido;
each m independently is 0, 1, or 2;
each R¹⁰ is the same Of different and is independently selected from alkylene, cycloalkylene, alkenylene, cycloalkenylene, and alkynylene;
each of R² and R³ are the same or different and are independently selected from the group consisting of H, alkyl, alkenyl, alkynyl, cycloalkyl, cycloalkenyl, —R¹⁰cycloalkyl, —R¹⁰OH, —R¹⁰(OR¹⁰)_{w}, and —R¹⁰NR⁴R⁵;
w is 1-10;
each of R⁴ and R⁵ are the same or different and are independently selected from the group consisting of alkyl, cycloalkyl, alkenyl, cycloalkenyl, and alkynyl;
Ay represents an aryl group; Het represent a 5- or 6-membered heterocyclyl or heteroaryl group; ring A is aryl or heteroaryl; provided that when the A ring is aryl, t is 0, and Y is SO₂, then p is not 0;
including salt, solvates and physiologically functional derivatives thereof.

A number of RdRp inhibitors are known in the art and used for virus-specific, or broad spectrum virus inhibition. In some embodiments the RdRp inhibitor is a nucleoside/nucleotide analog. In some embodiments the RdRp inhibitor is a non-nucleoside/nucleotide inhibitor. In some embodiments the RdRp inhibitor is selective for a species, or for a class of RNA viruses. In some embodiments the RdRp inhibitor is broad spectrum and active against two or more classes of virus. In some embodiments the RdRp inhibitor is pyrazinecarboxamide derivative, ag, favipiravir. In some embodiments the RdRp inhibitor is a cytidine analog, e.g. 4-azidocytidine (R1479) and its prodrug balapiravir (R1626).

In some embodiments the combination of RdRp inhibitor with one or both of pyrimidine salvage inhibitor, such as CPU analog, and inhibitor of de novo pyrimidine synthesis, such as a DHODH inhibitor, improves the therapeutic index of the RdRp inhibitor, where the therapeutic index may be calculated as the LD₅₀/TD₅₀. The combination therapy may lower the EC₅₀ of the RdRp by 2-fold, 3-fold, 4-fold, 5-fold, 6-fold, 7-fold, 8-fold, 9-fold, 10-fold or more.

The invention provides compositions and methods for the administration of formulations of these active agents, as well as unit dose forms of the formulations suitable for administration to patients. In some embodiments the ratio of inhibitor of DHODH to CPU analog may range from about 5000:1, 500:1, 100:1, 50:1, 25:1, 10:1, 5:1, 2:1, 1:1, 1:2, 1:5, 1:10, 1:25, 1:50, 1:100, 1:500, 1:5000 by weight, or by molarity. The dose of RdRp inhibitor may be based on the therapeutic dose, and may be decreased relative to the dose as a single agent.

Also provided are methods of inhibiting viral infection in a subject by administering an effective dose of a combination therapy of the invention to a subject infected with a virus or at risk of virus infection, e.g. a person known to be exposed to a pathogenic virus. In some embodiments the virus infects a mammal. In some embodiments the virus infects humans. In some embodiments the virus is an RNA virus, e.g. a Group III, Group IV, or Group V of the Baltimore classification system of classifying viruses, which groups comprise single stranded RNA viruses, double stranded RNA viruses, and retroviruses.

The subject combination of active agents may be formulated or provided to a subject in need thereof in combination with one or more additional agents, e.g. deoxycytidine supplementation, interferon, ribavirin, and the like for treatment of viral infection.

These and other advantages, and features of the disclosure will become apparent to those persons skilled in the art upon reading the details of the compositions and methods of use, which are more fully described below.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures.

FIG. 1. De novo and salvage biosynthesis of pyrimidine nucleotides for host and viral RNA synthesis. GSK983 is a DHODH inhibitor. Genes that sensitize cells to GSK983 are highlighted in yellow boxes. Reactions shown with blue arrows comprise the de novo biosynthetic pathway, whereas those with red arrows comprise the salvage pathway.

FIG. 2. Effects of combining CPU and GSK983 on dengue virus replication and cell proliferation in the presence of exogenous uridine. CPU synergized with GSK983 to decrease DENV-2 titer and CPU-GSK983 combination exhibited minimal toxicity in cell proliferation. Error bars represent ±S.D. of two replicates.

FIG. 3. Synthesis of the uracil moiety analogs. a) Diethyl azodicarboxylate (DEAD), PPh₃, THF, 3a (69%), 3b (53%), 3c (36%), 3d (59%); b) NH₃, MeOH; c) HCl, THF. Total yields for steps b and c: 4a (40%), 4b (60%), 4c (49%), 4d (36%). TBDPS=tert-butyldiphenyl silyl; Bz=Benzoyl.

FIG. 4. Synthesis of the cyclopentenyl fragment analogs. a, TBAF, THF; b: Ag₂O, Mel, acetone; c, NH₃, MeOH; d, HCl, THF; e, DAST, CH₂Cl₂; f, MsCl, Et₃N, CH₂Cl₂; g, NaN₃, DMF; h, AcCl, CH₂Cl₂; i, Pd(OH)₂, cyclohexene, ethanol. TBAF=Tetra-n-butylammonium fluoride, DAST=Diethylaminosulfur trifluoride, Ms=Methanesulfonyl, Ac=acetyl.

FIG. 5. Enzymatic analysis of CPU analogs. (A) CPU and 5-F-CPU are UCK2 substrates, as observed by addition of 250 μM of each CPU analogue to a UCK2 reaction mixture that also contains 50 μM uridine. All other analogs tested are not UCK2 substrates. (B) Steady-state kinetic analysis of uridine, CPU and 5-F-CPU as UCK2 substrates. Error bars represent ±S.D. of three replicates. (C) Comparative ATP consumption by UCK2 in the presence of 50 μM uridine and varying concentrations of CPU (blue bars) or 5-F-CPU (red bars). Error bars represent ±S.D. of three replicates. (D) Initial velocities calculated from the data shown in panel (A). (E) and (F) Lineweaver-Burk analysis of 5 -F-CPU and 5 -deoxy-CPU as UCK2 inhibitors.

FIG. 6. Comparative activity of CMPK1 on CMP, CPU-MP and 5-F-CPU-MP. An authentic standard of CMP (std) was tested alongside a UCK-synthesized sample of the same compound (syn). Error bars represent ±S.D. of three replicates. In vitro enzyme activity assays with recombinant human CMPK1: In a similar manner as UCK2 assay, ATP hydrolysis was coupled to NADH oxidation via pyruvate kinase (PK) and lactate dehydrogenase (LDH) to continuously monitor reaction progress spectrophotometrically. Reactions were conducted at room temperature in 50 μL in 96-well plates (Greiner Bio-One, UV-Star, Half Area). Mixtures contained 50 mM Tris HCl (pH 7.5), 50 mM KCl, 5 mM MgCl₂, 2 mM DTT, 500 μNADH, 1 mM PEP, 20 units/mL of PK and LDH. To 39.5 μL above reaction mixtures were add 5 μL 0-1 mM stock solutions of substrates (CMP from the commercial vendor and denatured UCK2 reaction mixtures containing newly synthesized CMP, CPU-MP and 5-F-CPUMP) in UCK2 assay buffer [20 mM HEPES (pH 7.2), 100 mM KCl, 2 mM MgCl2]. After the UV-readout at 340 nm reached stable in ca. 5 minutes, a mixture of ATP and CMPK1 in assay buffer [50 mM TrisHCl (pH 7.5), 50 mM KCl, 5 mM MgCl₂, 2 mM DTT] was added to give a final reaction volume of 50 μL and a final ATP concentration of 500 μM and CMPK1 of 20-100 nM. Progress was monitored in the linear region using a Biotek Synergy HT, and kinetic constants were determined using GraphPad Prism 7 (GraphPad Software).

FIG. 7. LC-MS analysis of intracellular nucleotides. (A) LC-MS analysis of intracellular uridine and cytidine nucleotide levels after 6 h treatments. In all assays, the culture medium was supplemented with 5 μM uridine and 1 μM GSK983. Error bars represent ±S.D. of two replicates. (B) LC-MS detection of mono-, di- and tri-phosphates of CPU and 5-F-CPU in cells. LC-MS/MS was performed on an Agilent 1290 infinity II LC system tandem with 6370 triple quad mass spectrometer using ion pairing chromatography. LC separation was performed at 40° C. with a solvent flow rate of 0.25 mL/min on a Zorbax RRHD Extended-C18 column (2.1×150 mm, 1.8 μM). Buffer A contained 97% H2O, 3% MeOH, 5 mM tributylamine, 5.5 mM Acetic acid and 1 μM medronic acid with pH=5.0; Buffer B contains ca. 100% MeOH, 5 mM tributylamine, 5.5 mM Acetic acid and 5 μM medronic acid with pH=7.0 The initial mobile phase composition was 100% solvent A, and a constant or linear gradient was applied after sample injection with eluent B varying as follows: 0% at 2.5 min, 20% at 7.5 min, 45% at 14 min, 99% at 20 to 23 min, and 0% from 23.1 to 27.1 min. The autosampler temperature was set to 4° C. and the sample was injected at 5 μL. Samples were measured in the negative ESI mode with capillary voltage at −3.5 kV. Further source settings were as follows: gas temperature, 250° C.; gas flow, 13 L/min; nebulizer, 35 psi; sheath gas temperature, 325° C.; sheath gas flow, 12 L/min; nozzle voltage, 500 V; and delta EMV, −200. Acquisition was performed in dynamic multiple reaction monitoring (dMRM) mode with fragmentation pattern and retention time setting as Table 1.

FIG. 8. Antiviral and cytotoxic activities of selected CPU analogs in combination with GSK983 in the presence of exogenous uridine. Effect of 1 μM GSK983 and 500 μM of individual CPU analogs on the viability of A549 cells (blue) and on replication of luciferase-expressing DENV-2 virus (red). Cell viability was measured via CellTiter-Glo luminescence assay system and virus titer was measured with Luciferase-Glo assay system. Error bars represent ±S.D. of three replicates.

FIG. 9. Improving the therapeutic window of RdRp inhibitor R1479 by combination treatment targeting pyrimidine biosynthesis with GSK983, CPU and 5-F-CPU. In all assays, the culture medium was supplemented with 20 μM uridine to mimic plasma uridine concentration and 1 μM GSK983 to block de nova pyrimidine biosynthesis unless otherwise specified. Control*: The dose-response curve of DENV-2 replication by R1479 without supplement of GSK983. Error bars represent ±S.D. of three replicates.

FIG. 10. Identities of CPU-MP and 5-F-CPU-MP, synthesized by UCK2 catalyzed mono-phosphorylation of CPU and 5-F-CPU, were confirmed by LC-MS/MS. TIC: Total Ion Chromatogram; MRM: Multiple Reaction Monitoring.

FIG. 11. LC-MS/MS monitoring of CMPK1-catalyzed conversion of CPU-MP and 5-F-CPU-MP to their corresponding diphosphates.

FIG. 12. Flow chart of cell sample preparation for metabolite analysis by LC-MS/MS. Preparation of cell lysis for LC-MS/MS analysis: A549 cells were plated overnight at 120,000 cells/well in 24-well plates in complete DMEM. The next day, cells were treated with 250 μM CPU, 5-F-CPU or 5′-F-CPU in DMEM additionally supplemented with 1 μM GSK983 and 5 μM uridine. Six hours after drug addition, the cell culture medium was removed and the whole plate was rapidly rinsed twice by dipping vertically into a beaker containing 37° C. Milli-Q water. The plate was then placed on dry ice, followed by the addition of 0.5 mL −20° C. lysis buffer (MeCN:MeOH:H₂O=2:2:1) containing 0.5 M formic acid and 10 nM 5,6-d-uridine (Santa Cruz Biotechnology). The solution was then sonicated for 3×5 minutes on ice for the metabolite extraction. Subsequently, the sample was frozen with liquid nitrogen, freeze-dried, re-suspended with 150 μL Milli-Q water, and finally filtered through a MultiScreen 96-well filter plate prior to LC-MS analysis.

FIG. 13. Addition of 1 μM GSK and 250 μM CPU displayed minimal impact on the cytotoxicity profile of R1479. Error bars represent ±S.D. of three replicates.

### DEFINITIONS

Before embodiments of the present disclosure are further described, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Any methods and materials similar or equivalent to those described herein can also be used in the practice or testing of embodiments of the present disclosure.

It must be noted that as used herein and in the appended claims, the singular forms “a”, “and”, and “the” include plural referents unless the context clearly dictates otherwise. Thus, for example, reference to “a compound” includes not only a single compound but also a combination of two or more compounds, reference to “a substituent” includes a single substituent as well as two or more substituents, and the like.

In describing and claiming the present invention, certain terminology will be used in accordance with the definitions set out below. It will be appreciated that the definitions provided herein are not intended to be mutually exclusive. Accordingly, some chemical moieties may fall within the definition of more than one term.

As used herein, the phrases “for example,” “for instance,” “such as,” or “including” are meant to introduce examples that further clarify more general subject matter. These examples are provided only as an aid for understanding the disclosure, and are not meant to be limiting in any fashion.

The terms “active agent,” “antagonist”, “inhibitor”, “drug” and “pharmacologically active agent” are used interchangeably herein to refer to a chemical material or compound which, when administered to an organism (human or animal) induces a desired pharmacologic and/or physiologic effect by local and/or systemic action.

As used herein, the terms “treatment,” “treating,” and the like, refer to obtaining a desired pharmacologic and/or physiologic effect, such as reduction of viral titer. The effect may be prophylactic in terms of completely or partially preventing a disease or symptom thereof and/or may be therapeutic in terms of a partial or complete cure for a disease and/or adverse effect attributable to the disease. “Treatment,” as used herein, covers any treatment of a disease in a mammal, particularly in a human, and includes: (a) preventing the disease or a symptom of a disease from occurring in a subject which may be predisposed to the disease but has not yet been diagnosed as having it (e.g., including diseases that may be associated with or caused by a primary disease; (b) inhibiting the disease, i.e., arresting its development; and (c) relieving the disease, i.e., causing regression of the disease (e.g., reduction in viral titers).

The terms “individual,” “host,” “subject,” and “patient” are used interchangeably herein, and refer to an animal, including, but not limited to, human and non-human primates, including simians and humans; rodents, including rats and mice; bovines; equines; ovines; felines; canines; avians, and the like. “Mammal” means a member or members of any mammalian species, and includes, by way of example, canines; felines; equines; bovines; ovines; rodentia, etc. and primates, e.g., non-human primates, and humans. Non-human animal models, e.g., mammals, e.g. non-human primates, murines, lagomorpha, etc. may be used for experimental investigations.

As used herein, the terms “determining,” “measuring,” “assessing,” and “assaying” are used interchangeably and include both quantitative and qualitative determinations.

The terms “polypeptide” and “protein”, used interchangeably herein, refer to a polymeric form of amino acids of any length, which can include coded and non-coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and native leader sequences, with or without N-terminal methionine residues; immunologically tagged proteins; fusion proteins with detectable fusion partners, e.g., fusion proteins including as a fusion partner a fluorescent protein, β-galactosidase, luciferase, etc.; and the like.

The terms “nucleic acid molecule” and “polynucleotide” are used interchangeably and refer to a polymeric form of nucleotides of any length, either deoxyribonucleotides or ribonucleotides, or analogs thereof. Polynucleotides may have any three-dimensional structure, and may perform any function, known or unknown. Non-limiting examples of polynucleotides include a gene, a gene fragment, exons, introns, messenger RNA (mRNA), transfer RNA, ribosomal RNA, ribozymes, cDNA, recombinant polynucleotides, branched polynucleotides, plasmids, vectors, isolated DNA of any sequence, control regions, isolated RNA of any sequence, nucleic acid probes, and primers. The nucleic acid molecule may be linear or circular.

A “therapeutically effective amount” or “efficacious amount” means the amount of a compound that, when administered to a mammal or other subject for treating a disease, condition, or disorder, is sufficient to effect such treatment for the disease, condition, or disorder. The “therapeutically effective amount” will vary depending on the compound, the disease and its severity and the age, weight, etc., of the subject to be treated.

A therapeutically effective dose of an agent also refers to that amount of the agent that results in amelioration of symptoms or a prolongation of survival in a subject. Toxicity and therapeutic efficacy of such molecules can be determined by standard pharmaceutical procedures in cell cultures or experimental animals, e.g., by determining the LD50 (the dose lethal to 50% of the population) and the ED50 (the dose therapeutically effective in 50% of the population). The dose ratio of toxic to therapeutic effects is the therapeutic index, which can be expressed as the ratio LD50/ED50. The therapeutic index of an RdRp inhibitor can be improved by co-administration with one or both of a CPU analog, e.g. as disclosed herein, and an inhibitor of DHODH.

The term “unit dosage form,” as used herein, refers to physically discrete units suitable as unitary dosages for human and animal subjects, each unit containing a predetermined quantity of a compound calculated in an amount sufficient to produce the desired effect in association with a pharmaceutically acceptable diluent, carrier or vehicle. The specifications for unit dosage forms depend on the particular compound employed and the effect to be achieved, and the pharmacodynamics associated with each compound in the host.

A “pharmaceutically acceptable excipient,” “pharmaceutically acceptable diluent,” “pharmaceutically acceptable carrier,” and “pharmaceutically acceptable adjuvant” means an excipient, diluent, carrier, and adjuvant that are useful in preparing a pharmaceutical composition that are generally safe, non-toxic and neither biologically nor otherwise undesirable, and include an excipient, diluent, carrier, and adjuvant that are acceptable for veterinary use as well as human pharmaceutical use. “A pharmaceutically acceptable excipient, diluent, carrier and adjuvant” as used in the specification and claims includes both one and more than one such excipient, diluent, carrier, and adjuvant.

As used herein, a “pharmaceutical composition” is meant to encompass a composition suitable for administration to a subject, such as a mammal, especially a human. In general a “pharmaceutical composition” is sterile, and preferably free of contaminants that are capable of eliciting an undesirable response within the subject (e.g., the compound(s) in the pharmaceutical composition is pharmaceutical grade). Pharmaceutical compositions can be designed for administration to subjects or patients in need thereof via a number of different routes of administration including oral, buccal, rectal, parenteral, intraperitoneal, intradermal, intracheal, intramuscular, subcutaneous, and the like.

Pathogenic virus. The compositions and methods of the present invention provide for improved treatment of pathogenic viruses, particularly viruses that infect avians and mammals for medical and veterinary use. Viruses include those that infect, e.g. farm animals including horses, cattle, sheep, pigs, chickens, turkeys, etc., domestic animals including dogs and cats; and viruses that infect humans, particularly RNA viruses.

An RNA virus is a virus that has RNA (ribonucleic acid) as its genetic material. This nucleic acid is usually single-stranded RNA (ssRNA) but may be double-stranded RNA (dsRNA). Human diseases caused by RNA viruses include, inter alia, AIDS, Ebola hemorrhoragic fever, SARS, influenza, hepatitis C, dengue fever, zika virus disease, West Nile fever, polio, and measles.

The ICTV classifies RNA viruses as those that belong to Group III, Group IV or Group V of the Baltimore classification system of classifying viruses and does not consider viruses with DNA intermediates in their life cycle as RNA viruses. Viruses with RNA as their genetic material but that include DNA intermediates in their replication cycle are retroviruses, and comprise Group VI of the Baltimore classification. Notable human retroviruses include HIV-1 and HIV-2, the cause of the disease AIDS. For the purposes of the present invention, an RNA virus is one that is within Group III, IV, V or VI unless otherwise indicated.

The double-stranded (ds)RNA viruses represent a diverse group of viruses that vary widely in host range, genome segment number, and virion organization. Members of this group include the rotaviruses and picobirnaviruses. The clades include the Caliciviridae, Flaviviridae, and Picornaviridae families, and a second that includes the Alphatetraviridae, Birnaviridae and Cystoviridae, Nodaviridae, and Permutotretraviridae families. Double-stranded RNA viruses (Group III) contain from one to a dozen different RNA molecules, each coding for one or more viral proteins.

RNA viruses can be further classified according to the sense or polarity of their RNA into negative-sense and positive-sense, or ambisense RNA viruses. Positive-sense ssRNA viruses (Group IV) have their genome directly utilized as if it were mRNA, with host ribosomes translating it into a single protein that is modified by host and viral proteins to form the various proteins needed for replication. One of these includes RNA-dependent RNA polymerase (RNA replicase), which copies the viral RNA to form a double-stranded replicative form. In turn this directs the formation of new virions. Viruses in this group include I. Bymoviruses, comoviruses, nepoviruses, nodaviruses, picornaviruses, potyviruses, sobemoviruses and a subset of luteoviruses (beet western yellows virus and potato leaf roll virus)-the picorna like group (Picornavirata); II. Carmoviruses, dianthoviruses, flaviviruses, pestiviruses, tombusviruses, hepatitis C virus and a subset of luteoviruses (barley yellow dwarf virus)-the flavi like group (Flavivirata); Ill. Alphaviruses, carlaviruses, furoviruses, hordeiviruses, potexviruses, rubiviruses, tobraviruses, tricornaviruses, tymoviruses and hepatitis E virus-the alpha like group (Rubivirata). Alphaviruses and flaviviruses can be separated into two families-the Togaviridae and Flaviridae. Coronavirus are of particular interest, e.g. SARS-CoV1, SARS-CoV2; MERS-CoV, etc.

Negative-sense ssRNA viruses (Group V) must have their genome copied by an RNA replicase to form positive-sense RNA. The positive-sense RNA molecule then acts as viral mRNA, which is translated into proteins by the host ribosomes. The resultant protein goes on to direct the synthesis of new virions, such as capsid proteins and RNA replicase, which is used to produce new negative-sense RNA molecules. Group V-negative-sense ssRNA viruses include one order and eight families in this group. The group includes a number of clinically relevant pathogens. Bornaviridae-Borna disease virus; Family Filoviridae-includes Ebola virus, Marburg virus; Family Paramyxoviridae-includes Measles virus, Mumps virus, Nipah virus, Hendra virus, RSV and NDV; Family Rhabdoviridae—includes Rabies virus; Family Nyamiviridae-includes Nyavirus; Family Arenaviridae-includes Lassa virus; Family Bunyaviridae-includes Hantavirus, Crimean-Congo hemorrhagic fever; Family Ophioviridae; Family Orthomyxoviridae-includes Influenza viruses; Genus Deltavirus—includes Hepatitis D virus; Genus Dichorhavirus; Genus Emaravirus; Genus Nyavirus—includes Nyamanini and Midway viruses; Genus Tenuivirus; Genus Varicosavirus

Retroviruses (Group VI) have a single-stranded RNA genome although they use DNA intermediates to replicate. Reverse transcriptase, a viral enzyme that comes from the virus itself after it is uncoated, converts the viral RNA into a complementary strand of DNA, which is copied to produce a double-stranded molecule of viral DNA. After this DNA is integrated into the host genome using the viral enzyme integrase, expression of the encoded genes may lead to the formation of new virions. Included in retroviruses are the lentiviruses, e.g. HIV-1 and HIV-2.

“In combination with”, “combination therapy” and “combination products” refer, in certain embodiments, to the concurrent administration to a patient of a first therapeutic and the compounds as used herein. When administered in combination, each component can be administered at the same time or sequentially in any order at different points in time. Thus, each component can be administered separately but sufficiently closely in time so as to provide the desired therapeutic effect.

RdRp inhibitors. RNA dependent RNA polymerase (RdRp) is an enzyme of RNA viruses that is required for replicating the genome and carrying out transcription. The core structural features of RdRps are conserved between viruses. RdRps are multi-domain (α and β) proteins belonging to Structural Classification of Proteins (SCOP) class 2.7.7.48. They catalyze RNA-template dependent formation of phosphodiester bonds between ribonucleotides in the presence of divalent metal ions. The initiation of synthesis occurs at the 3′-end of the template in a primer-dependent or independent manner and proceeds in the 5′→3′ direction. The average length of the core RdRp domain is less than 500 amino acids and is folded into three subdomains, viz., thumb, palm, and fingers resembling a right-handed cup. The active sites of RdRps from different RNA viruses are conserved and show resemblances to those of other enzymes such as reverse transcriptases and DNA polymerases indicating their similar role in nucleotidyl transfer reactions.

Many viral polymerases possess additional domains such as methyltransferase or endonuclease domain to carry out functions associated with RNA synthesis. The polymerase domain may also interact with other host factors for efficient polymerization and to discriminate activities such as genome replication and mRNA transcription. The host factors include translation factors, protein chaperones, RNA-modifying enzymes, and a few other cellular proteins. These together with the RdRps, constitute the viral replication complexes (VRCs).

A number of RdRp inhibitors are known in the art and can be used in the methods of the invention. RdRp inhibitors can be specific for a virus, or class of viruses. Examples of broad spectrum inhibitors are described by Furuta et al. Proc Jpn Acad Ser B Phys Biol Sci. 2017; 93(7):449-463, including Favipiravir (T-705), a broad spectrum inhibitor effective against a wide range of types and subtypes of influenza viruses, including strains resistant to existing anti-influenza drugs, and other RNA viruses such as arenaviruses, bunyaviruses and filoviruses. Madhvi et al. Sci Rep. 2017 Jul. 19; 7(1):5816 describe the broad-spectrum antiviral compound NSC-320218 as a potent inhibitor against HCV, dengue virus and hepatitis E virus.

Other inhibitors include, without limitation, inhibitors of calicivirus, including norovirus, in Netzler et al. Antiviral Res. 2017 October; 146:65-75; Tarantino et al. Antiviral Res. 2014 Feburary; 102:23-8 pyridoxal-5′-phosphate-6-(2′-naphthylazo-6′-nitro-4′,8′-disulfonate) tetrasodium salt (PPNDS); and Eltahla et al. Antimicrob Agents Chemother. 2014 June; 58(6):3115-23.

Dengue virus RdRp inhibitors include Celgosivir, NITD-008, NITD107, and Balapiravir; non-nucleoside inhibitors described in Lim et al. Adv Exp Med Biol. 2018; 1062:187-198; functionalized 2,1-benzothiazine 2,2-dioxides by Cannalire et al. Eur J Med Chem. 2018 January 1; 143:1667-1676; 5(1H)-Quinazolinone,2-(4-bromophenyl)-2,3,4,6,7,8-hexahydro-7,7-dimethyl-1,3-diphenyl (Q63) by Yao et al. J Pharmacol Sci. 2018 December; 138(4):247-256; compounds disclosed by Yokohawa et al. J Med Chem. 2016 Apr. 28; 59(8):3935-52; Manvar et al. Biochem Biophys Res Commun. 2016 Jan. 15; 469(3):743-7, conjugated thiazolidinone-thiadiazole scaffold by Pelliccia et al. J Enzyme Inhib Med Chem. 2017 December; 32(1):1091-1101.

Alphaviruses include Hepatitis C virus, for which a number of RdRp inhibitors have been described, including sofosbuvir, dasabuvir; cyclopropylindolobenzazepine inhibitors described by Rahman et al. Mol Biosyst. 2016 October 18; 12(11):3280-3293; Meguellati et al. Eur J Med Chem. 2016 June 10; 115:217-29; pseudodimeric aurones; Paparin et al. Bioorg Med Chem Lett. 2017 Jun. 1; 27(11):2634-2640 describes the benzophosphadiazine drug candidate IDX375. R1479 (4′-azidocytidine) is exemplified herein as an RdRp inhibitor of HCV.

Zika virus RdRp is inhibited by sofosbuvir, and pyridoxine-derived non-nucleoside small-molecule inhibitor, DMB213, discussed by Xu et al. J Antimicrob Chemother. 2017 Mar. 1; 72(3):727-734. Elf iky et al. SAR QSAR Environ Res. 2018 May; 29(5):409-418 teaches the use of Setrobuvir, YAK and IDX-184.

Inhibitors of influenza virus RdRp include 2-oxo-pyrazine-3-carboxamide-yl nucleoside analogues; 4-[(1S, 3R, 4R, 7R)-7-hydroxy-1-(hydroxymethyl)-2,5-dioxabicyclo[2.2.1]heptan-3-yl]-3-oxo-3,4-dihydropyrazine-2-carboxamide 8a; Lepri et al. J Med Chem. 2014 May 22; 57(10):4337-50 disclose thiophene-3-carboxamide to polyamido scaffolds; and compounds described by Abdel-Magid ACS Med Chem Lett. 2013 Oct. 18; 4(12):1133-4.

Inhibitors of respiratory syncytial virus (RSV) RdRp include PC786, disclosed by Coates et al. Antimicrob Agents Chemother. 2017 Aug. 24; 61(9); and ALS-8112 and ALS-8176 disclosed by Deval et al. PLoS One, 2016 May 10; 11(5):e0154097.

Dihydroorotate dehydrogenase (DHODH) catalyzes the fourth enzymatic step, the ubiquinone-mediated oxidation of dihydroorotate to orotate, in de novo pyrimidine biosynthesis. This protein is a mitochondrial protein located on the outer surface of the inner mitochondrial membrane. The enzyme classification is EC 1.3.3.1. Human DHODH has two domains: an alpha/beta-barrel domain containing the active site and an alpha-helical domain that forms the opening of a tunnel leading to the active site.

For use in the present invention, inhibitors of DHODH include known inhibitors as well as compounds that are identified herein as inhibitors. Known inhibitors of DHODH include the immunomodulatory drugs teriflunomide, leflunomide and brequinar. Other inhibitors include, without limitation, those disclosed in, for example Baumgartner et al. (2006) J. Med. Chem. 49(4):1239-1247; Lolli et al. (2012) Eur. J. Med. Chem. 49:102-109; Lucas-Hourani et al. (2015) J. Med. Chem. 58(14):5579-5598. Included in such compositions are GSK983, a tetrahydrocarbazole that inhibits the replication of a variety of unrelated viruses in vitro with EC₅₀ values of 5-20 nM (see Harvey et al. (2009) Antiviral Res. 82(1):1-11) and analogs thereof. Such analogs may have the structure:

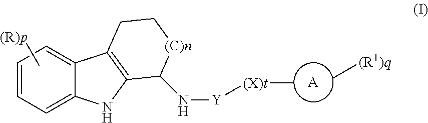

wherein: n is 0, 1, or 2; tis 0 or 1; X is —NH—, —O—, —R¹⁰—, —OR¹⁰—, —R¹⁰O—, —R¹⁰OR¹⁰—, —NR¹⁰—, —R¹⁰N—, —R¹⁰NR¹⁰—, —R¹⁰ S(O)ₘ—, or —R¹⁰S(O)ₘR¹⁰—; Y is —C(O)— or —S(O)ₘ—;
each R is the same or different and is independently selected from the group consisting of halogen, haloalkyl, alkyl, alkenyl, alkynyl, cycloalkyl, cycloalkenyl, —R¹⁰cycloalkyl, Ay, —NHR¹⁰Ay, Het, —NHHet, —NHR¹⁰Het, —OR², —OAy, —OHet, —R¹⁰OR², —NR²R³, —NR²Ay, —R¹⁰NR²R³, —R¹⁰NR²Ay, —R¹⁰C(O)R², —C(O)R², —CO₂R², —R¹⁰CO₂R², —C(O)NR²R³, —C(O)Ay, —C(O)NR²Ay, —C(O)Het, —C(O)NHR¹⁰Het, —R¹⁰C(O)NR²R³, —C(S)NR²R³, —R¹⁰C(S)NR²R³, —R¹⁰NHC(NH)NR²R³, —C(NH)NR²R³, —R¹⁰C(NH)NR²R³, —S(O)₂NR²R³, —S(O)₂NR²Ay, —R¹⁰SO₂NHCOR², —R¹⁰SO₂NR²R³, —R¹⁰SO₂R², —S(O)ₘR², —S(O)ₘAy, cyano, nitro, or azido;
each R¹ is the same or different and is independently selected from the group consisting of halogen, haloalkyl, alkyl, alkenyl, alkynyl, cycloalkyl, cycloalkenyl, —R¹⁰cycloalkyl, Ay, —NHR¹⁰Ay, Het, —NHHet, —NHR¹⁰Het, —OR², —OAy, —OHet, —R¹⁰OR², —NR²R³, —NR²Ay, —R¹⁰NR²R³, —R¹⁰NR²Ay, —R¹⁰C(O)R², —C(O)R², —CO₂R², —R¹⁰CO₂R², —C(O)NR²R³, —C(O)Ay, —C(O)NR²Ay, —C(O)Het, —C(O)NHR¹⁰Het, —R¹⁰C(O)NR²R³, —C(S)NR²R³, —R¹⁰C(S)NR²R³, —R¹⁰NHC(NH)NR²R³, —C(NH)NR²R³, —R¹⁰C(NH)NR²R³, —S(O)₂NR²R³, —S(O)₂NR²Ay, —R¹⁰SO₂NHCOR², —R¹⁰SO₂NR²R³, —R¹⁰SO₂R², —S(O)ₘR², —S(O)ₘAy, cyano, nitro, or azido;
each m independently is 0, 1, or 2;
each R¹⁰ is the same or different and is independently selected from alkylene, cycloalkylene, alkenylene, cycloalkenylene, and alkynylene;
p and q are each independently selected from 0, 1, 2, 3, 4, or 5;
each of R² and R³ are the same or different and are independently selected from the group consisting of H, alkyl, alkenyl, alkynyl, cycloalkyl, cycloalkenyl, —R¹⁰cycloalkyl, —R¹⁰H, —R¹⁰(OR¹⁰)_{w}, and —R¹⁰NR⁴R⁵;
w is 1-10;
each of R⁴ and R⁵ are the same or different and are independently selected from the group consisting of alkyl, cycloalkyl, alkenyl, cycloalkenyl, and alkynyl;
Ay represents an aryl group; Het represents a 5- or 6-membered heteracyclyl or heteroaryl group; ring A is aryl or heteroaryl; provided that when the A ring is aryl, t is 0, and Y is SO₂, then p is not 0;
including salts, solvates and physiologically functional derivatives thereof.

In some embodiments the inhibitor of DHODH is GSK983 or an analog thereof, including without limitation 6Br-pF, 6Br-oTol, and GSK984, which compounds have the following structures:

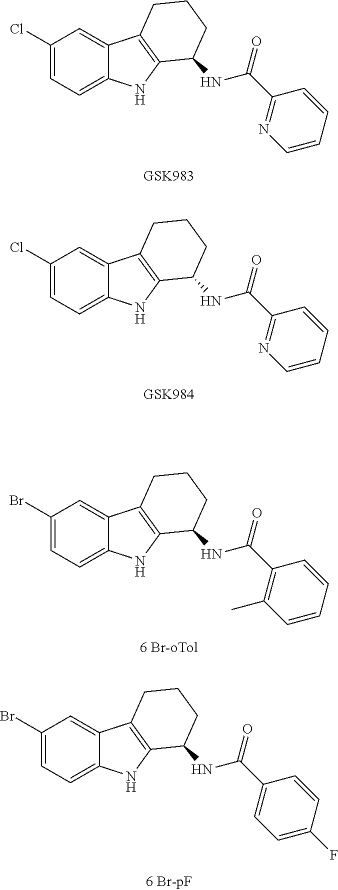 [SMILES: O=C(N[C@@H]1CCCc2c1[nH]c1ccc(Cl)cc21)c1ccccn1 ; O=C(N[C@H]1CCCc2c1[nH]c1ccc(Cl)cc21)c1ccccn1 ; Cc1ccccc1C(=O)N[C@@H]1CCCc2c1[nH]c1ccc(Br)cc21 ; O=C(N[C@@H]1CCCc2c1[nH]c1ccc(Br)cc21)c1ccc(F)cc1]

Pyrimidine salvage pathway. In pyrimidine salvage reactions, nucleosides and free bases generated by DNA and RNA breakdown are converted back to nucleotide monophosphates, allowing them to re-enter the pathways of pyrimidine biosynthesis. An inhibitor, which may be a selective inhibitor, of a protein in the pyrimidine salvage or nucleoside transport pathways may be referred to as a pyrimidine salvage pathway inhibitor generically, or as an inhibitor of the specific enzyme or transporter, e.g. a UCK2 inhibitor. Of specific interest are CPU analogs.

In some embodiments a CPU analog has a structure selected from the following group:

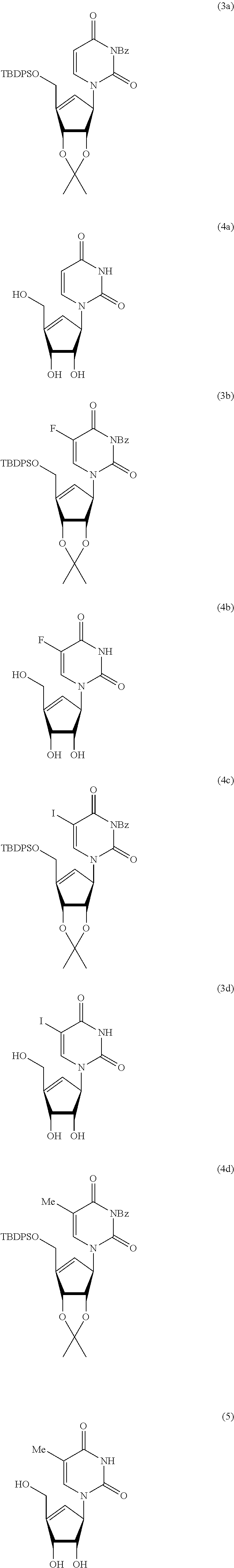

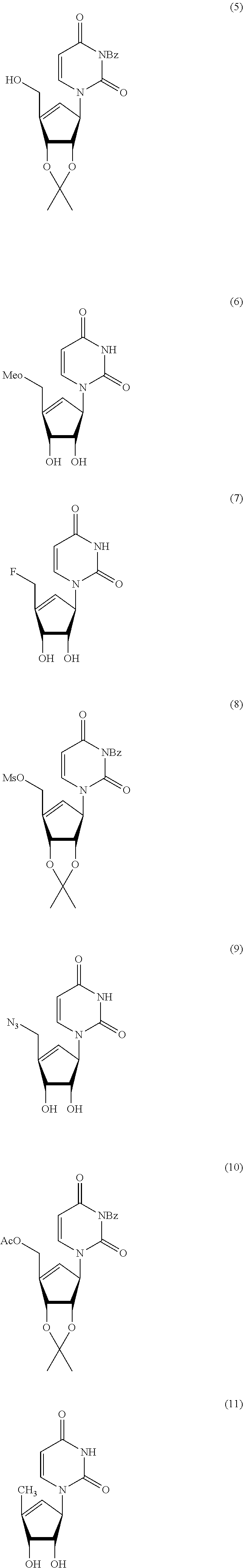

In some embodiments the CPU analog is one or both of ((3aS,4R,6aR)-4-(3-benzoyl-2,4-dioxo-3,4-dihydropyrimidin-1(2H)-yl)-2,2-dimethyl-3a,6a-dihydro-4H-cyclopenta[d][1,3]dioxol-6-yl)methyl methanesulfonate (structure 8 above), and 1-((1R,4R,5S)-4,5-dihydroxy-3-(hydroxymethyl)cyclopent-2-en-1-yl)-5-fluoropyrimidine-2,4(1H,3H)-dione (structure 4b above).

In some embodiments a CPU analog has a structure selected from the following group:

\textcircled{B} has a structure selected from the following group:

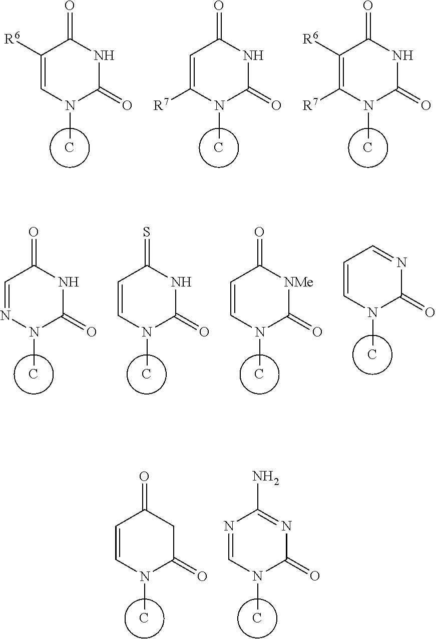

\textcircled{C} has a structure selected from the following group:

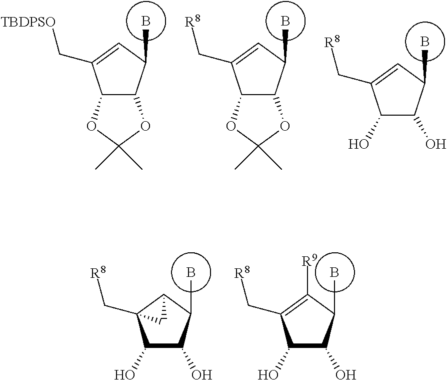

R⁶, R⁷, R^{,} and R⁹ is the same or different and is independently selected from the group consisting of halogen, haloalkyl, alkyl, alkenyl, alkynyl, cycloalkyl, cycloalkenyl, —R¹⁰cycloalkyl, Ay, —NHR¹⁰Ay, Het, —NHHet, —NHR¹⁰Het, —OR², —OAy, —OHet, —R¹⁰OR², —NR²R³, —NR²Ay, —R¹⁰NR²R³, —R¹⁰NR²Ay, —R¹⁰C(O)R², —C(O)R², —CO₂R², —R¹⁰CO₂R², —C(O)NR²R³, —C(O)Ay, —C(O)NR²Ay, —C(O)Het, —C(O)NHR¹⁰Het, —R¹⁰C(O)NR²R³, —C(S)NR²R³, —R¹⁰C(S)NR²R³, —R¹⁰NHC(NH)NR²R³, —C(NH)NR²R³, —R¹⁰C(NH)NR²R³, —S(O)₂NR²R³, —S(O)₂NR²Ay, —R¹⁰SO₂NHCOR², —R¹⁰SO₂NR²R³, —R¹⁰SO₂R², —S(O)ₘR², —S(O)ₘAy, cyano, nitro, or azido;
each m independently is 0, 1, or 2;
each R¹⁰ is the same or different and is independently selected from alkylene, cycloalkylene, alkenylene, cycloalkenylene, and alkynylene;
each of R² and R³ are the same or different and are independently selected from the group consisting of H, alkyl, alkenyl, alkynyl, cycloalkyl, cycloalkenyl, —R¹⁰cycloalkyl, —R¹⁰H, —R¹⁰(OR¹⁰)_{w}, and —R¹⁰NR⁴R⁵;
w is 1-10;
each of R⁴ and R⁵ are the same or different and are independently selected from the group consisting of alkyl, cycloalkyl, alkenyl, cycloalkenyl, and alkynyl;
Ay represents an aryl group; Het represents a 5- or 6-membered heteracyclyl or heteroaryl group; ring A is aryl or heteroaryl; provided that when the A ring is aryl, t is 0, and Y is SO₂, then p is not 0;
including salts, solvates and physiologically functional derivatives thereof.

The CPU analog can act synergistically with a DHODH inhibitor, alone or in combination with an RdRp inhibitor, to achieve improved activity against RNA viruses.

“In combination with”, “combination therapy” and “combination products” refer, in certain embodiments, to the concurrent administration to a patient of a first therapeutic and a second therapeutic, as used herein. When administered in combination, each component can be administered at the same time or sequentially in any order at different points in time. Thus, each component can be administered separately but sufficiently closely in time so as to provide the desired therapeutic effect. Alternatively, a co-formulation can include an effective dose of each of the active agents in a single formulation.

“Concomitant administration” of two or three active agents as set forth in the present invention means administration with the agents such that they will have a therapeutic effect. Such concomitant administration may involve concurrent (i.e. at the same time), prior, or subsequent administration of one agent with respect to the administration of the other agent. A person of ordinary skill in the art would have no difficulty determining the appropriate timing, sequence and dosages of administration for particular drugs and compositions of the present invention.

As used herein, endpoints for treatment will be given a meaning as known in the art and as used by the Food and Drug Administration.

Overall survival is defined as the time from randomization until death from any cause, and is measured in the intent-to-treat population. Survival is considered the most reliable endpoint, and when studies can be conducted to adequately assess survival, it is usually the preferred endpoint. This endpoint is precise and easy to measure, documented by the date of death. Bias is not a factor in endpoint measurement. Survival improvement should be analyzed as a risk-benefit analysis to assess clinical benefit. Overall survival can be evaluated in randomized controlled studies. Demonstration of a statistically significant improvement in overall survival can be considered to be clinically significant if the toxicity profile is acceptable and has often supported new drug approval. A benefit of the methods of the invention can include increased survival of patients with reduced toxicity relative to administration of the DHODH inhibitor as a single agent.

As used herein, the term “correlates,” or “correlates with,” and like terms, refers to a statistical association between instances of two events, where events include numbers, data sets, and the like. For example, when the events involve numbers, a positive correlation (also referred to herein as a “direct correlation”) means that as one increases, the other increases as well. A negative correlation (also referred to herein as an “inverse correlation”) means that as one increases, the other decreases.

“Dosage unit” refers to physically discrete units suited as unitary dosages for the particular individual to be treated. Each unit can contain a predetermined quantity of active compound(s) calculated to produce the desired therapeutic effect(s) in association with the required pharmaceutical carrier. The specification for the dosage unit forms can be dictated by (a) the unique characteristics of the active compound(s) and the particular therapeutic effect(s) to be achieved, and (b) the limitations inherent in the art of compounding such active compound(s).

“Pharmaceutically acceptable excipient” means an excipient that is useful in preparing a pharmaceutical composition that is generally safe, non-toxic, and desirable, and includes excipients that are acceptable for veterinary use as well as for human pharmaceutical use. Such excipients can be solid, liquid, semisolid, or, in the case of an aerosol composition, gaseous.

“Pharmaceutically acceptable salts and esters” means salts and esters that are pharmaceutically acceptable and have the desired pharmacological properties. Such salts include salts that can be formed where acidic protons present in the compounds are capable of reacting with inorganic or organic bases. Suitable inorganic salts include those formed with the alkali metals, e.g. sodium and potassium, magnesium, calcium, and aluminum. Suitable organic salts include those formed with organic bases such as the amine bases, e.g., ethanolamine, diethanolamine, triethanolamine, tromethamine, N methylglucamine, and the like. Such salts also include acid addition salts formed with inorganic acids (e.g., hydrochloric and hydrobromic acids) and organic acids (e.g., acetic acid, citric acid, maleic acid, and the alkane- and arene-sulfonic acids such as methanesulfonic acid and benzenesulfonic acid). Pharmaceutically acceptable esters include esters formed from carboxy, sulfonyloxy, and phosphonoxy groups present in the compounds, e.g., C₁₋₆ alkyl esters. When there are two acidic groups present, a pharmaceutically acceptable salt or ester can be a mono-acid-mono-salt or ester or a di-salt or ester; and similarly where there are more than two acidic groups present, some or all of such groups can be salified or esterified. Compounds named in this invention can be present in unsalified or unesterified form, or in salified and/or esterified form, and the naming of such compounds is intended to include both the original (unsalified and unesterified) compound and its pharmaceutically acceptable salts and esters. Also, certain compounds named in this invention may be present in more than one stereoisomeric form, and the naming of such compounds is intended to include all single stereoisomers and all mixtures (whether racemic or otherwise) of such stereoisomers.

The terms “pharmaceutically acceptable”, “physiologically tolerable” and grammatical variations thereof, as they refer to compositions, carriers, diluents and reagents, are used interchangeably and represent that the materials are capable of administration to or upon a human without the production of undesirable physiological effects to a degree that would prohibit administration of the composition.

As used herein, the phrase “having the formula” or “having the structure” is not intended to be limiting and is used in the same way that the term “comprising” is commonly used. The term “independently selected from” is used herein to indicate that the recited elements, e.g., R groups or the like, can be identical or different.

As used herein, the terms “may,” “optional,” “optionally,” or “may optionally” mean that the subsequently described circumstance may or may not occur, so that the description includes instances where the circumstance occurs and instances where it does not. For example, the phrase “optionally substituted” means that a non-hydrogen substituent may or may not be present on a given atom, and, thus, the description includes structures wherein a non-hydrogen substituent is present and structures wherein a non-hydrogen substituent is not present.

The term “alkyl” as used herein refers to a branched or unbranched saturated hydrocarbon group (i.e., a mono-radical) typically although not necessarily containing 1 to about 24 carbon atoms, such as methyl, ethyl, n-propyl, isopropyl, n-butyl, isobutyl, t-butyl, octyl, decyl, and the like, as well as cycloalkyl groups such as cyclopentyl, cyclohexyl and the like. Generally, although not necessarily, alkyl groups herein may contain 1 to about 18 carbon atoms, and such groups may contain 1 to about 12 carbon atoms. The term “lower alkyl” intends an alkyl group of 1 to 6 carbon atoms. “Substituted alkyl” refers to alkyl substituted with one or more substituent groups, and this includes instances wherein two hydrogen atoms from the same carbon atom in an alkyl substituent are replaced, such as in a carbonyl group (i.e., a substituted alkyl group may include a —C(═O)— moiety). The terms “heteroatom-containing alkyl” and “heteroalkyl” refer to an alkyl substituent in which at least one carbon atom is replaced with a heteroatom, as described in further detail infra. If not otherwise indicated, the terms “alkyl” and “lower alkyl” include linear, branched, cyclic, unsubstituted, substituted, and/or heteroatom-containing alkyl or lower alkyl, respectively.

The term “alkenyl” as used herein refers to a linear, branched or cyclic hydrocarbon group of 2 to about 24 carbon atoms containing at least one double bond, such as ethenyl, n-propenyl, isopropenyl, n-butenyl, isobutenyl, octenyl, decenyl, tetradecenyl, hexadecenyl, eicosenyl, tetracosenyl, and the like. Generally, although again not necessarily, alkenyl groups herein may contain 2 to about 18 carbon atoms, and for example may contain 2 to 12 carbon atoms. The term “lower alkenyl” intends an alkenyl group of 2 to 6 carbon atoms. The term “substituted alkenyl” refers to alkenyl substituted with one or more substituent groups, and the terms “heteroatom-containing alkenyl” and “heteroalkenyl” refer to alkenyl in which at least one carbon atom is replaced with a heteroatom. If not otherwise indicated, the terms “alkenyl” and “lower alkenyl” include linear, branched, cyclic, unsubstituted, substituted, and/or heteroatom-containing alkenyl and lower alkenyl, respectively.

The term “alkynyl” as used herein refers to a linear or branched hydrocarbon group of 2 to 24 carbon atoms containing at least one triple bond, such as ethynyl, n-propynyl, and the like. Generally, although again not necessarily, alkynyl groups herein may contain 2 to about 18 carbon atoms, and such groups may further contain 2 to 12 carbon atoms. The term “lower alkynyl” intends an alkynyl group of 2 to 6 carbon atoms. The term “substituted alkynyl” refers to alkynyl substituted with one or more substituent groups, and the terms “heteroatom-containing alkynyl” and “heteroalkynyl” refer to alkynyl in which at least one carbon atom is replaced with a heteroatom. If not otherwise indicated, the terms “alkynyl” and “lower alkynyl” include linear, branched, unsubstituted, substituted, and/or heteroatom-containing alkynyl and lower alkynyl, respectively.

The term “alkoxy” as used herein intends an alkyl group bound through a single, terminal ether linkage; that is, an “alkoxy” group may be represented as —O-alkyl where alkyl is as defined above. A “lower alkoxy” group intends an alkoxy group containing 1 to 6 carbon atoms, and includes, for example, methoxy, ethoxy, n-propoxy, isopropoxy, t-butyloxy, etc. Substituents identified as “C1-C6 alkoxy” or “lower alkoxy” herein may, for example, may contain 1 to 3 carbon atoms, and as a further example, such substituents may contain 1 or 2 carbon atoms (i.e., methoxy and ethoxy).

The term “aryl” as used herein, and unless otherwise specified, refers to an aromatic substituent generally, although not necessarily, containing 5 to 30 carbon atoms and containing a single aromatic ring or multiple aromatic rings that are fused together, directly linked, or indirectly linked (such that the different aromatic rings are bound to a common group such as a methylene or ethylene moiety). Aryl groups may, for example, contain 5 to 20 carbon atoms, and as a further example, aryl groups may contain 5 to 12 carbon atoms. For example, aryl groups may contain one aromatic ring or two or more fused or linked aromatic rings (i.e., biaryl, aryl-substituted aryl, etc.). Examples include phenyl, naphthyl, biphenyl, diphenylether, diphenylamine, benzophenone, and the like. “Substituted aryl” refers to an aryl moiety substituted with one or more substituent groups, and the terms “heteroatom-containing aryl” and “heteroaryl” refer to aryl substituent, in which at least one carbon atom is replaced with a heteroatom, as will be described in further detail infra. Aryl is intended to include stable cyclic, heterocyclic, polycyclic, and polyheterocyclic unsaturated C₃-C₁₄ moieties, exemplified but not limited to phenyl, biphenyl, naphthyl, pyridyl, furyl, thiophenyl, imidazoyl, pyrimidinyl, and oxazoyl; which may further be substituted with one to five members selected from the group consisting of hydroxy, C₁-C₈ alkoxy, C₁-C₈ branched or straight-chain alkyl, acyloxy, carbamoyl, amino, N-acylamino, nitro, halogen, trifluoromethyl, cyano, and carboxyl (see e.g. Katritzky, Handbook of Heterocyclic Chemistry). If not otherwise indicated, the term “aryl” includes unsubstituted, substituted, and/or heteroatom-containing aromatic substituents.

The term “aralkyl” refers to an alkyl group with an aryl substituent, and the term “alkaryl” refers to an aryl group with an alkyl substituent, wherein “alkyl” and “aryl” are as defined above. In general, aralkyl and alkaryl groups herein contain 6 to 30 carbon atoms. Aralkyl and alkaryl groups may, for example, contain 6 to 20 carbon atoms, and as a further example, such groups may contain 6 to 12 carbon atoms.

The term “alkylene” as used herein refers to a di-radical alkyl group. Unless otherwise indicated, such groups include saturated hydrocarbon chains containing from 1 to 24 carbon atoms, which may be substituted or unsubstituted, may contain one or more alicyclic groups, and may be heteroatom-containing. “Lower alkylene” refers to alkylene linkages containing from 1 to 6 carbon atoms. Examples include, methylene (—CH2—), ethylene (—CH2CH2-), propylene (—CH2CH2CH2-), 2-methylpropylene (—CH2-CH(CH3)-CH2-), hexylene (—(CH2)6-) and the like.

Similarly, the terms “alkenylene,” “alkynylene,” “arylene,” “aralkylene,” and “alkarylene” as used herein refer to di-radical alkenyl, alkynyl, aryl, aralkyl, and alkaryl groups, respectively.

The term “amino” is used herein to refer to the group —NRR′ wherein R and R′ are independently hydrogen or nonhydrogen substituents, with nonhydrogen substituents including, for example, alkyl, aryl, alkenyl, aralkyl, and substituted and/or heteroatom-containing variants thereof.

The terms “halo” and “halogen” are used in the conventional sense to refer to a chloro, bromo, fluoro or iodo substituent.

The term “heteroatom-containing” as in a “heteroatom-containing alkyl group” (also termed a “heteroalkyl” group) or a “heteroatom-containing aryl group” (also termed a “heteroaryl” group) refers to a molecule, linkage or substituent in which one or more carbon atoms are replaced with an atom other than carbon, e.g., nitrogen, oxygen, sulfur, phosphorus or silicon, typically nitrogen, oxygen or sulfur. Similarly, the term “heteroalkyl” refers to an alkyl substituent that is heteroatom-containing, the terms “heterocyclic” or “heterocycle” refer to a cyclic substituent that is heteroatom-containing, the terms “heteroaryl” and “heteroaromatic” respectively refer to “aryl” and “aromatic” substituents that are heteroatom-containing, and the like. Examples of heteroalkyl groups include alkoxyaryl, alkylsulfanyl-substituted alkyl, N-alkylated amino alkyl, and the like. Examples of heteroaryl substituents include pyrrolyl, pyrrolidinyl, pyridinyl, quinolinyl, indolyl, furyl, pyrimidinyl, imidazolyl, 1,2,4-triazolyl, tetrazolyl, etc., and examples of heteroatom-containing alicyclic groups are pyrrolidino, morpholino, piperazino, piperidino, tetrahydrofuranyl, etc.

As used herein, the terms “Heterocycle,” “heterocyclic,” “heterocycloalkyl,” and “heterocyclyl” refer to a saturated or unsaturated group having a single ring or multiple condensed rings, including fused bridged and spiro ring systems, and having from 3 to 15 ring atoms, including 1 to 4 hetero atoms. These ring atoms are selected from the group consisting of nitrogen, sulfur, or oxygen, wherein, in fused ring systems, one or more of the rings can be cycloalkyl, aryl, or heteroaryl, provided that the point of attachment is through the non-aromatic ring. In certain embodiments, the nitrogen and/or sulfur atom(s) of the heterocyclic group are optionally oxidized to provide for the N-oxide, —S(O)—, or —SO₂— moieties.

Examples of heterocycle and heteroaryls include, but are not limited to, azetidine, pyrrole, imidazole, pyrazole, pyridine, pyrazine, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, 1,2,3,4-tetrahydroisoquinoline, 4,5,6,7-tetrahydrobenzo[b]thiophene, thiazole, thiazolidine, thiophene, benzo[b]thiophene, morpholinyl, thiomorpholinyl (also referred to as thiamorpholinyl), 1,1-dioxothiomorpholinyl, piperidinyl, pyrrolidine, tetrahydrofuranyl, and the like.

Unless otherwise constrained by the definition for the heterocyclic substituent, such heterocyclic groups can be optionally substituted with 1 to 5, or from 1 to 3 substituents, selected from alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl, —SO₂-heteroaryl, and fused heterocycle.

“Hydrocarbyl” refers to univalent hydrocarbyl radicals containing 1 to about 30 carbon atoms, including 1 to about 24 carbon atoms, further including 1 to about 18 carbon atoms, and further including about 1 to 12 carbon atoms, including linear, branched, cyclic, saturated and unsaturated species, such as alkyl groups, alkenyl groups, aryl groups, and the like. A hydrocarbyl may be substituted with one or more substituent groups. The term “heteroatom-containing hydrocarbyl” refers to hydrocarbyl in which at least one carbon atom is replaced with a heteroatom. Unless otherwise indicated, the term “hydrocarbyl” is to be interpreted as including substituted and/or heteroatom-containing hydrocarbyl moieties.

By “substituted” as in “substituted hydrocarbyl,” “substituted alkyl,” “substituted aryl,” and the like, as alluded to in some of the aforementioned definitions, is meant that in the hydrocarbyl, alkyl, aryl, or other moiety, at least one hydrogen atom bound to a carbon (or other) atom is replaced with one or more non-hydrogen substituents. Examples of such substituents include, without limitation, functional groups, and the hydrocarbyl moieties C1-C24 alkyl (including C1-C18 alkyl, further including C1-C12 alkyl, and further including C1-C6 alkyl), C2-C24 alkenyl (including C2-C18 alkenyl, further including C2-C12 alkenyl, and further including C2-C6 alkenyl), C2-C24 alkynyl (including C2-C18 alkynyl, further including C2-C12 alkynyl, and further including C2-C6 alkynyl), C5-C30 aryl (including C5-C20 aryl, and further including C5-C12 aryl), and C6-C30 aralkyl (including C6-C20 aralkyl, and further including C6-C12 aralkyl). The above-mentioned hydrocarbyl moieties may be further substituted with one or more functional groups or additional hydrocarbyl moieties such as those specifically enumerated. Unless otherwise indicated, any of the groups described herein are to be interpreted as including substituted and/or heteroatom-containing moieties, in addition to unsubstituted groups.

By the term “functional groups” is meant chemical groups such as halo, hydroxyl, sulfhydryl, C1-C24 alkoxy, C2-C24 alkenyloxy, C2-C24 alkynyloxy, C5-C20 aryloxy, acyl (including C2-C24 alkylcarbonyl (—CO-alkyl) and C6-C20 arylcarbonyl (—CO-aryl)), acyloxy (—O-acyl), C2-C24 alkoxycarbonyl (—(CO)—O-alkyl), C6-C20 aryloxycarbonyl (—(CO)—O-aryl), halocarbonyl (—CO)—X where X is halo), C2-C24 alkylcarbonato (—O—(CO)—O-alkyl), C6-C20 arylcarbonato (—O—(CO)—O-aryl), carboxy (—COOH), carboxylato (—COO—), carbamoyl (—(CO)—NH2), mono-substituted C1-C24 alkylcarbamoyl (—(CO)—NH(C1-C24 alkyl)), di-substituted alkylcarbamoyl (—(CO)—N(C1-C24 alkyl)2), mono-substituted arylcarbamoyl (—(CO)—NH-aryl), thiocarbamoyl (—(CS)—NH2), carbamido (—NH—(CO)—NH2), cyano (—C≡N), isocyano (—N+≡C—), cyanato (—O—C≡N), isocyanato (—O—N+≡C—), isothiocyanato (—S—C≡N), azido (—N═N+═N—), formyl (—(CO)—H), thioformyl (—(CS)—H), amino (—NH2), mono- and di-(C1-C24 alkyl)-substituted amino, mono- and di-(C5-C20 aryl)-substituted amino, C2-C24 alkylamido (—NH—(CO)-alkyl), C5-C20 arylamido (—NH—(CO)-aryl), imino (—CR═NH where R=hydrogen, C1-C24 alkyl, C5-C20 aryl, C6-C20 alkaryl, C6-C20 aralkyl, etc.), alkylimino (—CR═N(alkyl), where R=hydrogen, alkyl, aryl, alkaryl, etc.), arylimino (—CR═N(aryl), where R=hydrogen, alkyl, aryl, alkaryl, etc.), nitro (—NO2), nitroso (—NO), sulfo (—SO2-OH), sulfonato (—SO2-O—), C1-C24 alkylsulfanyl (—S-alkyl; also termed “alkylthio”), arylsulfanyl (—S-aryl; also termed “arylthio”), C1-C24 alkylsulfinyl (—(SO)-alkyl), C5-C20 arylsulfinyl (—(SO)-aryl), C1-C24 alkylsulfonyl (—SO2-alkyl), C5-C20 arylsulfonyl (—SO2-aryl), phosphono (—P(O)(OH)2), phosphonato (—P(O)(O—)2), phosphinato (—P(O)(O—)), phospho (—PO2), and phosphino (—PH2), mono- and di-(C1-C24 alkyl)-substituted phosphino, mono- and di-(C5-C20 aryl)-substituted phosphine. In addition, the aforementioned functional groups may, if a particular group permits, be further substituted with one or more additional functional groups or with one or more hydrocarbyl moieties such as those specifically enumerated above.

By “linking” or “linker” as in “linking group,” “linker moiety,” etc., is meant a bivalent radical moiety that connects two groups via covalent bonds. Examples of such linking groups include alkylene, alkenylene, alkynylene, arylene, alkarylene, aralkylene, and linking moieties containing functional groups including, without limitation: amido (—NH—CO—), ureylene (—NH—CO—NH—), imide (—CO—NH—CO—), epoxy (—O—), epithio (—S—), epidioxy (—O—O—), carbonyldioxy (—O—CO—O—), alkyldioxy (—O—(CH2)n-O—), epoxyimino (—O—NH—), epimino (—NH—), carbonyl (—CO—), etc. Any convenient orientation and/or connections of the linkers to the linked groups may be used.

When the term “substituted” appears prior to a list of possible substituted groups, it is intended that the term apply to every member of that group. For example, the phrase “substituted alkyl and aryl” is to be interpreted as “substituted alkyl and substituted aryl.”

In certain embodiments, a substituent may contribute to optical isomerism and/or stereo isomerism of a compound. Salts, solvates, hydrates, and prodrug forms of a compound are also of interest. All such forms are embraced by the present disclosure. Thus the compounds described herein include salts, solvates, hydrates, prodrug and isomer forms thereof, including the pharmaceutically acceptable salts, solvates, hydrates, prodrugs and isomers thereof. In certain embodiments, a compound may be a metabolized into a pharmaceutically active derivative.

Unless otherwise specified, reference to an atom is meant to include isotopes of that atom. For example, reference to H is meant to include 1H, 2H (i.e., D) and 3H (i.e., T), and reference to C is meant to include 12C and all isotopes of carbon (such as 13C).

Definitions of other terms and concepts appear throughout the detailed description below.

### DETAILED DESCRIPTION OF THE EMBODIMENTS

As summarized above, compounds and methods are provided for the treatment of pathogenic viral infections, particularly pathogenic RNA virus infections. The anti-infective combinations of the invention may have broad spectrum activity against a variety of virus infections. The anti-infective combinations may have an improved therapeutic index relative to, for example, the use of an RdRp inhibitor as a single agent.

Also provided are pharmaceutical compositions that include the subject combination formulations, where the combined active agents of the invention are formulated with a pharmaceutically acceptable excipient. Formulations may be provided in a unit dose, where the dose provides an amount of the compound or compounds effective to achieve a desired result, including without limitation inhibition of pathogenic RNA virus replication.

In some embodiments, the subject compounds, formulated separately or as a combination of agents, are provided by oral dosing and absorbed into the bloodstream. In some embodiments, the oral bioavailability of the subject compounds is 30% or more. Modifications may be made to the subject compounds or their formulations using any convenient methods to increase absorption across the gut lumen or their bioavailability.

In some embodiments, the subject compounds are metabolized in vivo to produce one or more metabolites. In some embodiments, the subject compounds may be optimized for metabolic stability using any convenient methods. In some embodiments, the subject compounds are metabolically stable (e.g., remain substantially intact in vivo during the half-life of the compound). In certain embodiments, the compounds have a half-life (e.g., an in vivo half-life) of 5 minutes or more, such as 10 minutes or more, 12 minutes or more, 15 minutes or more, 20 minutes or more, 30 minutes or more, 60 minutes or more, 2 hours or more, 6 hours or more, 12 hours or more, 24 hours or more, or even more.

In some embodiments, the subject compositions comprise an inhibitor of

DHODH, including without limitation GSK983 or an analog thereof, and a CPU analog as described herein. In some embodiments an inhibitor of RdRp is included in the combination. The activity of the active agents may be determined by an inhibition assay, e.g., by an assay that determines the level of activity of the enzyme either in a cell-free system or in a cell after treatment with a subject compound, relative to a control, by measuring the IC₅₀ or EC₅₀ value, respectively. In certain embodiments, the subject compounds have an IC₅₀ value (or EC₅₀ value) of 10 μM or less, such as 3 μM or less, 1 μM or less, 500 nM or less, 300 nM or less, 200 nM or less, 100 nM or less, 50 nM or less, 30 nM or less, 10 nM or less, 5 nM or less, 3 nM or less, 1 nM or less, or even lower.

In some embodiments, a CPU analog for use in the combinations of the invention has an activity as determined by a kinase activity assay, e.g., by an assay that determines the level of incorporation of radiolabeled phosphate from [γ-³²P]-ATP into a substrate molecule after treatment with a subject compound, relative to a control, by measuring the beta-particle emission rate using a scintillation counter or phosphorimaging. In certain embodiments, the subject compounds have an IC₅₀ value for UCK2 and/or CMPK1 of less than about 1 μM, less than about 0.2 μM, less than about 0.1 μM, less than about 10 nM, less than about 1 nM, or even less. The CPU analog may be selective for UCK2 and/or CMPK1.

In certain embodiments, the CPU analog has no significant effect on the viability of a mammalian cell, as determined by a cell cytotoxicity assay, e.g., as determined by administering a subject compound to a HeLa cell and determining the number of viable cells present. The subject compounds may exhibit a % cell viability, as compared to a control (e.g., a DMSO control), of 15% or more, such as 20% or more, 30% or more, 40% or more, 50% or more, 60% or more, 70% or more, 80% or more, 90% or more, 100% or more, 120% or more, or even higher. The subject compounds may exhibit a CC₅₀ value of 1 nM or higher, such as 100 nM or higher, 300 nM or higher, 1 μM or higher, 3 μM or higher, 5 μM or higher, 10 μM or higher, 20 μM or higher, 30 μM or higher, 50 μM or higher, or even higher. The combination of the CPU analog and the DHODH inhibitor may have a cellular toxicity profile that is substantially less toxic than the DHODH inhibitor administered as a single agent, e.g. a reduction in toxicity of at least about 50%, at least about 90%, at least about 99%, or more.

In certain embodiments, the combination of agents has a therapeutic index (e.g., the ratio of a compound's cytotoxicity (e.g., cell cytotoxicity, CC50) to bioactivity (e.g., antiviral activity, EC50)) that is 20 or more, such as 50 or more, 100 or more, 200 or more, 300 or more, 400 or more, 500 or more, or even more.

The therapeutic index of the RdRp inhibitor may be improved when administered in a combination therapy with a DHODH inhibitor and CPU analog. As an example, the EC50 of R1479 as a single agent is about 35 μM, but is lowered to about 8 μM or less in the combination therapy, therefore providing at least a 2-fold, at least a 3-fold, at least a 4-fold, or more improvement in therapeutic index.

The combination of agents may inhibit virus replication for a virus of interest by 10% to 100%, e.g., by 10% or more, 20% or more, 30% or more, 40% or more, 50% or more, 60% or more, 70% or more, 80% or more, or 90% or more. In certain assays, a subject combination of agents may inhibit its virus target with an IC₅₀ of 1×10⁻⁶ M or less (e.g., 1×10⁻⁶ M or less, 1×10⁻⁷ M or less, 1×10⁻⁸ M or less, 1×10⁻⁹ M or less, 1×10⁻¹⁰ M or less, or 1×10⁻¹¹ M or less).

The protocols that may be employed in determining activity are numerous, and include but are not limited to cell-free assays, e.g., binding assays; assays using purified enzymes, cellular assays in which a cellular phenotype is measured, e.g., gene expression assays; and in vivo assays that involve a particular animal (which, in certain embodiments may be an animal model for a condition related to the target pathogenic virus). Included for example is cytopathic effect (CPE) inhibition assay. CPE is morphological changes in cells caused by cytopathogenic virus infection. CPE assay is used to evaluate ability to inhibit CPE. Cell-based ELISA measures reduction of viral antigen in infected cells using anti-virus monoclonal antibody. The abundance of viral protein in infected cells treated with the combination is compared to that of the untreated control as a measure of antiviral activity. qPCR assay uses oligonucleotide primers and a probe amplifying virus-specific target sequence to detect the presence of virus nucleic acids. Reduction of virus nucleic acid in infected cells is used an indicator of antiviral efficacy. Plaque reduction assay measures the plaque forming efficiency of a virus in the presence of different concentrations of a test article. Yield reduction assay is a labor-intensive but powerful technique for evaluating a compound's antiviral efficacy. The three-step assay involves: infecting cells in the presence of different concentrations of the test article; collecting the cells or cell culture supernatants after a cycle of virus replication; and determining virus titers by plaque assay, TCID50, or quantitative real-time PCR. Hemagglutination-inhibition test (HAI) tests the efficacy of influenza drug candidates in preventing virus-induced hemagglutination.

### Methods

The present disclosure provides methods of treating pathogenic virus infection by targeting a combination of 2 or 3 host functions upon which the virus is dependent, thereby decreasing the ability of the virus to avoid the therapy by mutation. The methods also provide a broad platform for anti-infective therapies by targeting a host function.

Aspects of the method include contacting an individual or a cellular sample with a subject formulation (e.g., as described above) under conditions by which the formulation inhibits multiple pathways in both virus and in a mammalian host cell. Any convenient protocol for contacting the compound with the sample may be employed. The particular protocol that is employed may vary, e.g., depending on whether the sample is in vitro or in vivo. For in vitro protocols, contact of the sample with the compound may be achieved using any convenient protocol. In some instances, the sample includes cells that are maintained in a suitable culture medium, and the complex is introduced into the culture medium. For in vivo protocols, any convenient administration protocol may be employed. Depending upon the potency of the compound, the cells of interest, the manner of administration, the number of cells present, various protocols may be employed.

The term “sample” as used herein relates to a material or mixture of materials, typically, although not necessarily, in fluid form, containing one or more components of interest.

In some embodiments, the subject method is a method of treating a subject for an infective disease. In some embodiments, the subject method includes administering to the subject an effective amount of a formulation as described above. In some embodiments, the infective disease condition results from infection with a positive-stranded RNA virus, negative stranded RNA virus, or a dsRNA virus. In some embodiments, the infective disease condition results from infection with a pathogen selected from the group consisting of HCV, HIV1 , HIV2, rhinovirus (e.g., B or C), Ebola virus, hantavirus, Japanese encephalitis virus, hepatitis A virus, and influenza virus, Poliovirus, Enterovirus (e.g., A-D), West Nile Virus, and Dengue Virus (e.g., 1-4).

In some embodiments, the subject is human. In some embodiments, the compound is administered as a pharmaceutical preparation.

In some embodiments, where the subject method is a method of inhibiting viral infection, the method including contacting virus-infected cells with an effective dose of a combination described herein to inhibit viral replication. In some embodiments, the method further includes contacting the cells with an additional therapeutic agent, including without limitation deoxycytidine supplementation.

The subject compounds and methods find use in a variety of therapeutic applications. Therapeutic applications of interest include those applications in which pathogen infection is the cause or a compounding factor in disease progression. As such, the subject compounds find use in the treatment of a variety of different conditions in which the inhibition and/or treatment of viral infection in the host is desired.

### Pharmaceutical Compositions

The above-discussed compounds can be formulated using any convenient excipients, reagents and methods. Compositions are provided in formulation with a pharmaceutically acceptable excipient(s). A wide variety of pharmaceutically acceptable excipients are known in the art and need not be discussed in detail herein. Pharmaceutically acceptable excipients have been amply described in a variety of publications, including, for example, A. Gennaro (2000) “Remington: The Science and Practice of Pharmacy,” 20th edition, Lippincott, Williams, & Wilkins; Pharmaceutical Dosage Forms and Drug Delivery Systems (1999) H. C. Ansel et al., eds., 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

In some embodiments, the subject compound is formulated in an aqueous buffer.

Suitable aqueous buffers include, but are not limited to, acetate, succinate, citrate, and phosphate buffers varying in strengths from 5 mM to 100 mM. In some embodiments, the aqueous buffer includes reagents that provide for an isotonic solution. Such reagents include, but are not limited to, sodium chloride; and sugars e.g., mannitol, dextrose, sucrose, and the like. In some embodiments, the aqueous buffer further includes a non-ionic surfactant such as polysorbate 20 or 80. Optionally the formulations may further include a preservative. Suitable preservatives include, but are not limited to, a benzyl alcohol, phenol, chlorobutanol, benzalkonium chloride, and the like. In many cases, the formulation is stored at about 4° C. Formulations may also be lyophilized, in which case they generally include cryoprotectants such as sucrose, trehalose, lactose, maltose, mannitol, and the like. Lyophilized formulations can be stored over extended periods of time, even at ambient temperatures. In some embodiments, the subject compound is formulated for sustained release.

In some embodiments, the subject combination of agents is formulated with an additional antiviral agent, e.g. interferon, ribavirin, Enfuvirtide; RFI-641 (4,4″-bis-{4,6-bis-[3-(bis-carbamoylmethyl-sulfamoyl)-phenylamino]-(1,3,5) triazin-2-ylamino}-biphenyl-2,2″-disulfonic acid); BMS-433771 (2H-Imidazo(4,5-c)pyridin-2-one, 1-cyclopropyl-1,3-dihydro-3-((1-(3-hydroxypropyl)-1H-benzimidazol-2-yl)methyl)); arildone; Pleconaril (3-(3,5-Dimethyl-4-(3-(3-methyl-5-isoxazolyl)propoxy)phenyl)-5-(trifluoromethyl)-1,2,4-oxadiazole); Amantadine (tricyclo[3.3.1.1.3,7]decane-1-amine hydrochloride); Rimantadine (alpha-methyltricyclo[3.3.1.1.3,7]decane-1-methanamine hydrochloride); Acyclovir (acycloguanosine); Valaciclovir; Penciclovir (9-(4-hydroxy-3-hydroxymethyl-but-1-yl)guanine); Famciclovir (diacetyl ester of 9-(4-hydroxy-3-hydroxymethyl-but-1-yl)-6-deoxyguanine); Gancyclovir (9-(1,3-dihydroxy-2-propoxymethyl)guanine); Ara-A (adenosine arabinoside); Zidovudine (3′-azido-2′,3′-dideoxythymidine); Cidofovir (1-[(S)-3-hydroxy-2-(phosphonomethoxy)propyl]cytosine dihydrate); Dideoxyinosine (2′,3′-dideoxyinosine); Zalcitabine (2′,3′-dideoxycytidine); Stavudine (2′,3′-didehydro-2′,3′-dideoxythymidine); Lamivudine ((−)-β-L-3′-thia-2′,3′-dideoxycytidine); Abacavir (1S,4R)-4-[2-amino-6-(cyclopropylamino)-9H-purin-9-yl]-2-cyclopentene-1-methanol succinate); Emtricitabine (−)-β-L-3′-thia-2′,3′-dideoxy-5-fluorocytidine); Tenofovir disoproxil (Fumarate salt of bis(isopropoxycarbonyloxymethyl) ester of (R)-9-(2-phosphonylmethoxypropyl)adenine); Bromovinyl deoxyuridine (Brivudin); Iodo-deoxyuridine (Idoxuridine); Trifluorothymidine (Trifluridine); Nevirapine (11-cyclopropyl-5,11-dihydro-4-methyl-6H-dipyrido[3,2-b:2′,3′-f][1,4]diazepin-6-one); Delavirdine (1-(5-methanesulfonamido-1H-indol-2-yl-carbonyl)-4-[3-(1-methylethyl-amino)pyridinyl) piperazine monomethane sulfonated); Efavirenz ((−)6-chloro-4-cyclopropylethynyl-4-trifluoromethyl-1,4-dihydro-2H-3,1-benzoxazin-2-one); Foscarnet (trisodium phosphonoformate); Ribavirin (1-β-D-ribofuranosyl-1H-1,2,4-triazole-3-carboxamide); Raltegravir (N-[(4-Fluorophenyl)methyl]-1,6-dihydro-5-hydroxy-1-methyl-2-[1-methyl-1-[[(5-methyl-1,3,4-oxadiazol-2-yl)carbonyl]amino]ethyl]-6-oxo-4-pyrimidinecarboxamide monopotassium salt); Neplanocin A; Fomivirsen; Saquinavir (SQ); Ritonavir ([5S-(5R,8R,10R,11R)]-10-hydroxy-2-methyl-5-(1-methylethyl)-1-[2-(methylethyl)-4-thiazolyl]-3,6-dioxo-8,11-bis(phenylmethyl)-2,4,7,12-tetraazatridecan-13-oic acid 5-thiazolylmethyl ester); Indinavir ([(1S,2R,5(S)-2,3,5-trideoxy-N-(2,3-dihydro-2-hydroxy-1H-inden-1-yl)-5-[2-[[(1,1-dimethylethyl)amino]carbonyl]-4-pyridinylmethyl)-1-piperazinyl]-2-(phenylmethyl- -erythro)pentonamide); Amprenavir; Nelfinavir; Lopinavir; Atazanavir; Bevirimat; Indinavir; Relenza; Zanamivir; Oseltamivir; Tarvacin; etc. are administered to individuals in a formulation (e.g., in the same or in separate formulations) with a pharmaceutically acceptable excipient(s).

The subject formulations can be administered orally, subcutaneously, intramuscularly, parenterally, or other route, including, but not limited to, for example, oral, rectal, nasal, topical (including transdermal, aerosol, buccal and sublingual), vaginal, parenteral (including subcutaneous, intramuscular, intravenous and intradermal), intravesical or injection into an affected organ.

Each of the active agents can be provided in a unit dose of from about 0.1 μg, 0.5 μg, 1 μg, 5 μg, 10 μg, 50 μg, 100 μg, 500 μg, 1 mg, 5 mg, 10 mg, 50, mg, 100 mg, 250 mg, 500 mg, 750 mg or more. Administration may be every 4 hours, every 6 hours, every 12 hours, daily, every other day, weekly, or as empirically determined for the virus of interest and the host of interest.

The subject compounds may be administered in a unit dosage form and may be prepared by any methods well known in the art. Such methods include combining the subject compound with a pharmaceutically acceptable carrier or diluent which constitutes one or more accessory ingredients. A pharmaceutically acceptable carrier is selected on the basis of the chosen route of administration and standard pharmaceutical practice. Each carrier must be “pharmaceutically acceptable” in the sense of being compatible with the other ingredients of the formulation and not injurious to the subject. This carrier can be a solid or liquid and the type is generally chosen based on the type of administration being used.

Examples of suitable solid carriers include lactose, sucrose, gelatin, agar and bulk powders. Examples of suitable liquid carriers include water, pharmaceutically acceptable fats and oils, alcohols or other organic solvents, including esters, emulsions, syrups or elixirs, suspensions, solutions and/or suspensions, and solution and or suspensions reconstituted from non-effervescent granules and effervescent preparations reconstituted from effervescent granules. Such liquid carriers may contain, for example, suitable solvents, preservatives, emulsifying agents, suspending agents, diluents, sweeteners, thickeners, and melting agents. Preferred carriers are edible oils, for example, corn or canola oils. Polyethylene glycols, e.g. PEG, are also good carriers.

Any drug delivery device or system that provides for the dosing regimen of the instant disclosure can be used. A wide variety of delivery devices and systems are known to those skilled in the art.

### Subjects Amenable to Treatment Using the Compounds of the Disclosure

Individuals who have been clinically diagnosed as infected with a pathogen of interest are suitable for treatment with the methods of the present disclosure, as are individuals at risk of exposure. In particular embodiments of interest, individuals of interest for treatment according to the disclosure have detectable pathogen titer indicating active replication, for example a titer of at least about 10⁴, at least about 10⁵, at least about 5×10⁵, or at least about 10⁶, or greater than 2 million genome copies of virus per milliliter of serum. Similar methods may be used to determine whether subjects infected with another pathogen are suitable for treatment using the subject methods.

The effectiveness of the anti-infective treatment may be determined using any convenient method. For example, whether a subject method is effective in treating a virus infection can be determined by measuring viral load, or by measuring a parameter associated with infection.

Viral load can be measured by measuring the titer or level of virus in serum. These methods include, but are not limited to, a quantitative polymerase chain reaction (PCR) and a branched DNA (bDNA) test. Many such assays are available commercially, including a quantitative reverse transcription PCR (RT-PCR) (Amplicor HCV Monitor™, Roche Molecular Systems, New Jersey); and a branched DNA (deoxyribonucleic acid) signal amplification assay (Quantiplex™ HCV RNA Assay (bDNA), Chiron Corp., Emeryville, Calif.). See, e.g., Gretch et al. (1995) Ann. Intern. Med. 123:321-329.

### EXAMPLES

The following examples are put forth so as to provide those of ordinary skill in the art with a complete disclosure and description of how to make and use embodiments of the present disclosure, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present disclosure. All such modifications are intended to be within the scope of the claims appended hereto.

### Example 1

### Enhancing the Antiviral Efficacy of RNA-Dependent RNA Polymerase Inhibition by Combination With Modulators of Pyrimidine Metabolism

Genome-wide analysis of the mode of action of GSK983, a potent antiviral agent, led to the identification of dihydroorotate dehydrogenase (DHODH) as its target, along with the discovery that knockdown of uridine-cytidine kinase 2 (UCK2) or cytidine monophosphate kinase 1 (CMPK1) further sensitized cells to GSK983. To explore the pharmacological potential of this synthetic lethal relationship, we synthesized and evaluated analogs of cyclopentenyl uracil (CPU), an inhibitor of uridine salvage. Biochemical analysis revealed that CPU and 5-fluoro-CPU were substrates of UCK2, whereas 5′-fluoro-CPU and 5′-deoxy-CPU were inhibitors but not substrates. CPU and 5-fluoro-CPU monophosphates were also substrates of CMPK1. CPU and its monophosphate were better substrates of UCK2 and CMPK1, respectively, than the corresponding fluorinated analogs. Mass spectrometry confirmed that both CPU and 5-fluoro-CPU were converted into their corresponding mono-, di-, and tri-phosphate derivatives in cells, and that CPU addition led to a larger drop in the intracellular UTP and CTP pools. Consistent with all of this data, CPU showed greater synergy with GSK983 in dengue virus replication assays than any other analog tested. We hypothesized that this synergy depended on the exceptional ability of CPU to deplete UTP and CTP pools while simultaneously leading to the buildup of CPU triphosphate to levels that inhibit viral RNA-dependent RNA polymerase (RdRp). In support of this hypothesis, CPU and GSK983 markedly increased the potency of a RdRp inhibitor against dengue virus. Our findings highlight a new host-targeting strategy for potentiating the antiviral activities of RdRp inhibitors.

RNA virus infections cause serious diseases such as hepatitis, influenza, Ebola, dengue, and Lassa fever, yet many of them lack suitable antiviral treatments. We identified a host-targeting antiviral strategy by modulating pyrimidine metabolism with analogs of cyclopentenyl uracil (CPU), an inhibitor of pyrimidine salvage, and GSK983, an inhibitor of de novo biosynthesis. This combination therapy markedly increased the potency of R1479, an RNA-dependent RNA polymerase (RdRp) inhibitor, against dengue virus replication. At efficacious drug doses, the effect on the growth rates of uninfected cells was minimal. In light of the growing interest in RdRp inhibitors as antiviral agents, our findings shine light on a promising way to enhance their clinical utility by combining them with modulators of mammalian pyrimidine metabolism.

Significant progress in the development of antiviral drugs has come by targeting viral proteins with small molecules. For examples, compounds like aciclovir and zidovudine block viral reverse transcriptase to treat herpes simplex virus and HIV infections, respectively, and RNA-dependent RNA polymerase (RdRp) inhibitors like dasabuvir and sofosbuvir are used to treat hepatitis C virus infections. Meanwhile, targeting host proteins required for viral propagation is emerging as an attractive alternative that may circumvent the emergence of resistance. For example, maraviroc inhibits the human chemokine receptor CCR5, and is therefore used to treat multidrug-resistant HIV. More recently, pyrimidine biosynthesis has emerged as a potential host-targeting strategy for antivirals. Here, we focus on devising a host-targeting antiviral approach for the treatment of RNA viruses, which cause many serious diseases such as hepatitis, influenza, Ebola, dengue, and Lassa fever.

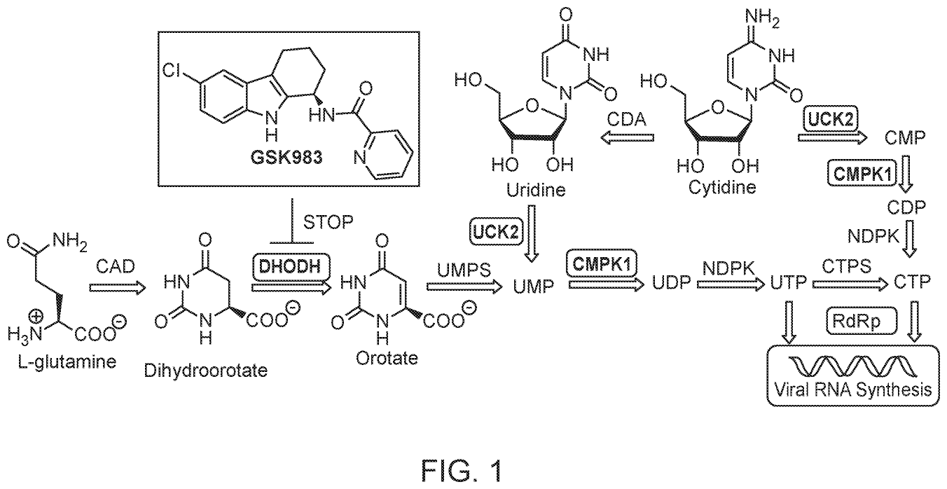

In mammalian cells, pyrimidine biosynthesis is a tightly regulated metabolic process. Two complementary pathways—de novo biosynthesis and pyrimidine salvage—are responsible for producing UTP and CTP for host as well as viral RNA synthesis (FIG. 1). De novo pyrimidine biosynthesis is a resource-intensive process. In contrast, salvage occurs via phosphorylation of UMP and CMP derived from intracellular RNA degradation or via facilitated transport and phosphorylation of extracellular uridine, whose plasma concentration is tightly controlled in the low micromolar range. Recently we discovered that GSK983, a broad-spectrum antiviral agent first reported in 2009, is a potent inhibitor of dihydroorotate dehydrogenase (DHODH), a rate-limiting step in de novo pyrimidine biosynthesis. In the course of those unbiased genome-wide studies, we also found that knockdown of uridine/cytidine kinase 2 (UCK2) and cytidine monophosphate kinase 1 (CMPK1) in the pyrimidine salvage pathway strongly sensitized cells to growth inhibition by GSK983. This finding was consistent with the observation that GSK983 lacks antiviral efficacy in vivo despite high potency in vitro presumably due to salvage metabolism of circulating uridine by virus-infected cells. To restore the antiviral efficacy of GSK983 in the presence of extracellular uridine, we therefore sought to inhibit UCK2 and/or CMPK1 based on their synthetic lethal relationship to DHODH. Cyclopentenyl uridine (CPU) is a carbocyclic analogue of uridine that has been shown to inhibit human UCK2. To our surprise, we learned that the antiviral activity of CPU is due to its remarkable ability to block multiple successive targets in the pyrimidine salvage and viral replication pathways. Our findings led us to redirect our search for a fundamentally new type of combination chemotherapy for RNA viruses, as described below.

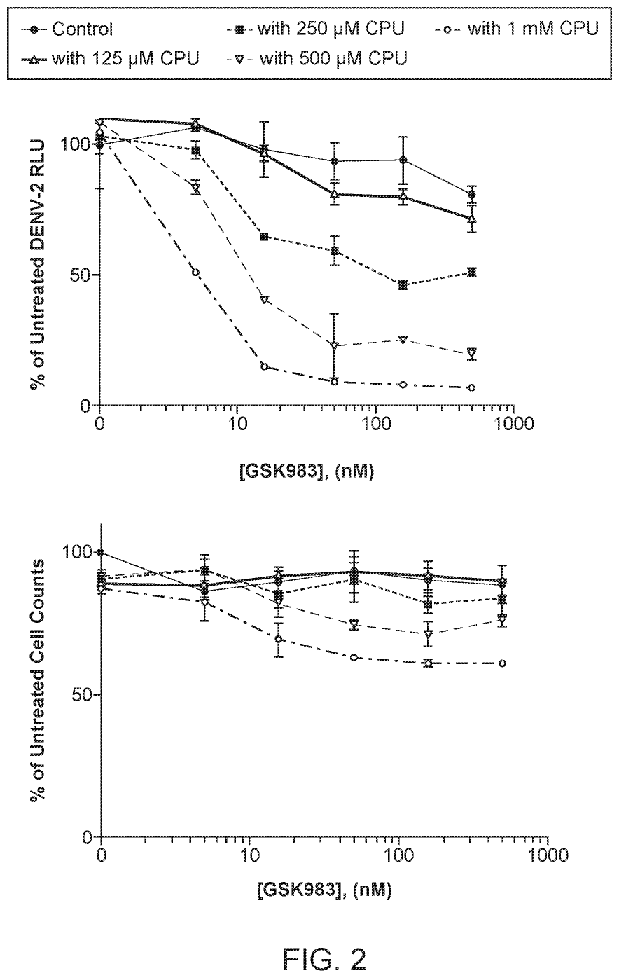

Our search for lead inhibitors of pyrimidine salvage was inspired by earlier reports on the biological activity of cyclopentenyl uracil (CPU) and cyclopentenyl cytosine (CPC), both of which were shown to block uridine salvage in vitro and in vivo. In those studies, CPU was found to be well-tolerated, whereas CPC was considerably more cytotoxic. We therefore focused our efforts on evaluating CPU in combination with GSK983. Using an infectious clone of dengue serotype 2 (DENV-2) strain 16681 engineered to express a luciferase reporter, the efficacy and cytotoxicity of combinations of GSK983 and CPU were assessed in infected or uninfected cultures of the A549 lung carcinoma cell line (FIG. 2). In the presence of 20 μM exogenous uridine, neither GSK nor CPU alone inhibited replication of DENV-2 virus. However, a combination of 0.2 μM GSK and 250 μM CPU inhibited ca. 50% of virus replication. At a CPU dose of 1 mM, virus replication was suppressed almost completely. Notably, the combination treatment had minimal effects on A549 cell growth, suggesting that combinations of GSK and CPU could be selective for inhibition of virus but not host replication.

Encouraged by the success of our preliminary experiments, we undertook structure-activity relationship (SAR) analysis of CPU. Due to the high toxicity of CPC, the uracil nucleobase was maintained intact or only modified at the C-5 position. Meanwhile, the C-5′ substituent of CPU was also modified, because this is the site of UCK2-catalyzed phosphorylation. In order to rapidly access both nucleobase and carbocyclic moiety analogs, we implemented a diversity-oriented synthetic approach featuring a Mitsunobu reaction as the strategic transformation.

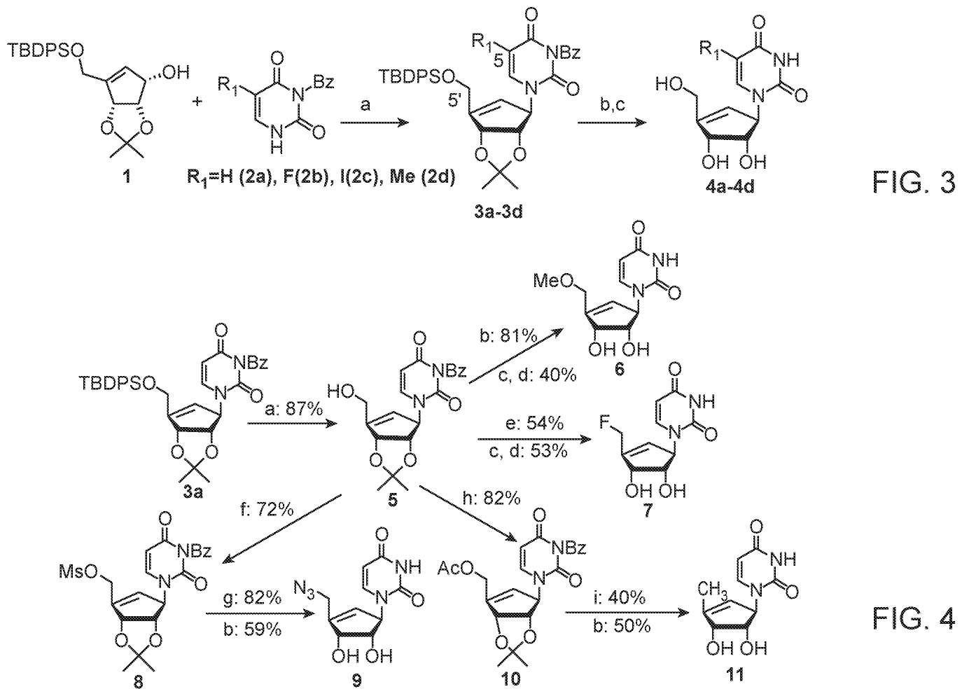

We first synthesized CPU analogs with nucleobase modifications (FIG. 3). Mitsunobu reactions between the common cyclopentenyl moiety 1 and the benzoyl protected uracil, C(5)-fluoro-uracil, C(5)-iodo-uracil or thymine (2a-2d) (15) furnished the carbon skeleton of C(5) analogs. Removal of acetal, benzoyl and TBDPS groups then delivered analogs 4a-4d.

Next, we turned to synthesis of CPU analogs modified at the C-5′ position of the cyclopentenyl moiety (FIG. 4). Selective deprotection of TBDPS with TBAF revealed the primary hydroxyl group, which was then methylated in the presence of Ag₂O to afford the protected 5′-methoxy-CPU in 81% yield. Alternatively, the alcohol could be converted to a terminal fluoride with diethylamino sulfurotrifluoride (DAST), resulting in protected 5′-fluoro CPU. Mesylation or acetylation of 5, followed by NaN3 substitution (19) or Pd(OH)₂/C catalyzed hydrogenation afforded the protected 5′-azido- and 5′-deoxy-CPU, respectively. The resulting protected intermediates were treated with methanolic ammonia and/or HCl to provide analogs 6, 7, 9 and 11.

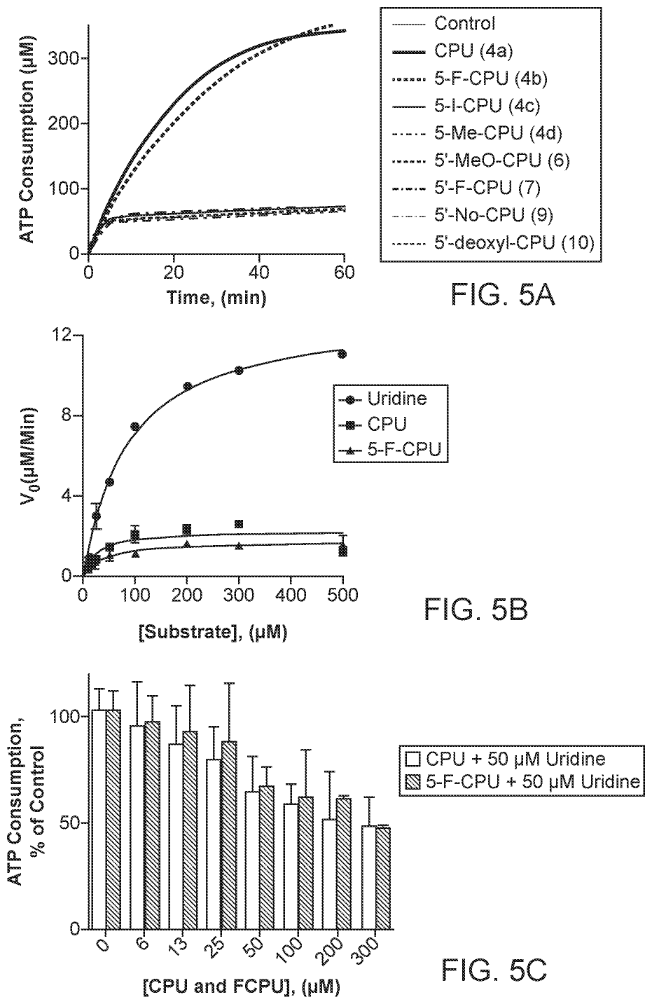

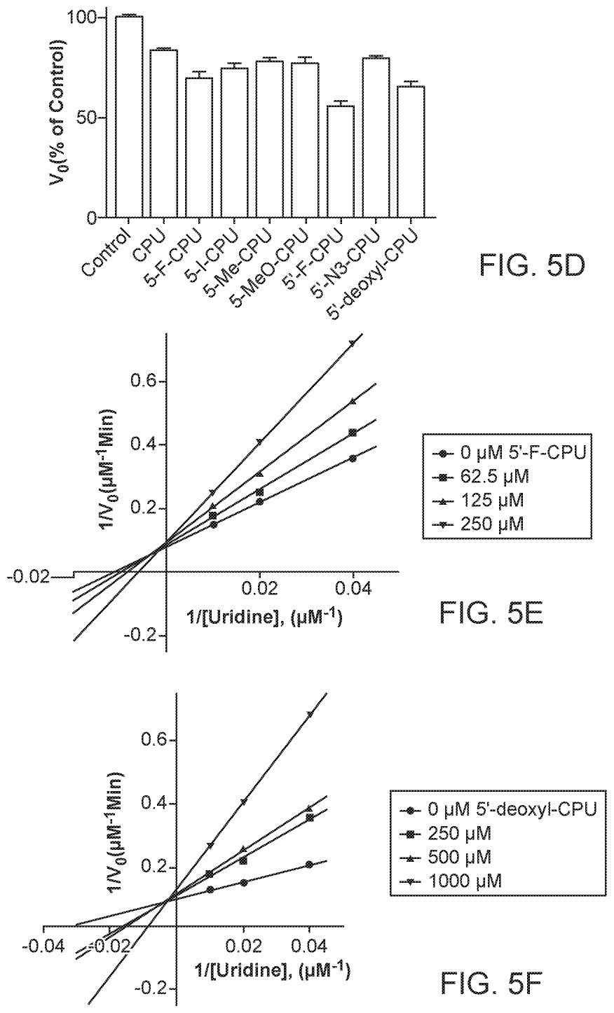

In order to guide our SAR studies, we developed an enzymatic assay to evaluate the inhibitory effect of each CPU analog against recombinant human UCK2, which was expressed and purified in E. coli. A continuous assay system was optimized by coupling UCK2 activity to the pyruvate kinase (PK) reaction, which in turn was coupled to lactate dehydrogenase (LDH). Overall reaction progress was continuously monitored by detecting the UV absorption change at 340 nm, which was correlated with the ATP consumption by UCK2. The effect of 250 μM of each CPU analog on UCK2 activity was evaluated in the presence of 50 μM uridine. CPU and 5-F-CPU showed much higher ATP consumption compared to other analogs (FIG. 5A), indicating these two compounds were substrates of UCK2. We then individually measured the steady state kinetic parameters of UCK2 using uridine, CPU and 5-F-CPU as substrates. Their KM values were 86, 25, and 41 μM, and their kcat/KM values were 2.6×10⁵, 1.5×10⁵, and 7.3×10⁴ s⁻¹ M⁻¹ (FIG. 5B). As predicted by the relative magnitude of these kinetic parameters, addition of CPU or 5-F-CPU to an assay mixture containing UCK2, uridine and ATP resulted in a dose-dependent decrease in the rate of UMP synthesis (FIG. 5C).

While CPU and 5-F-CPU were the only UCK2 substrates identified from our panel of carbocyclic nucleoside analogs, some of the other agents had measurable inhibitory activity against this enzyme (FIG. 5D). The Ki values of the two most potent competitive inhibitors, 5′-F-CPU and 5′-deoxy-CPU, were 170 μM and 230 μM, respectively (FIGS. 5 E and F).

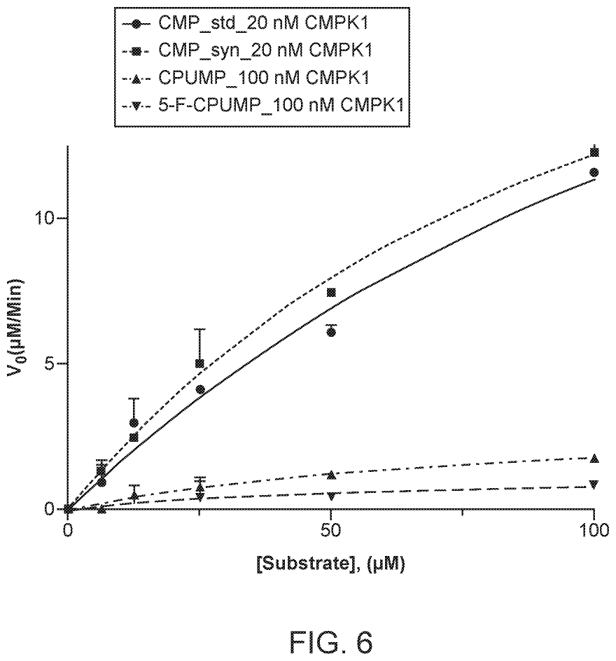

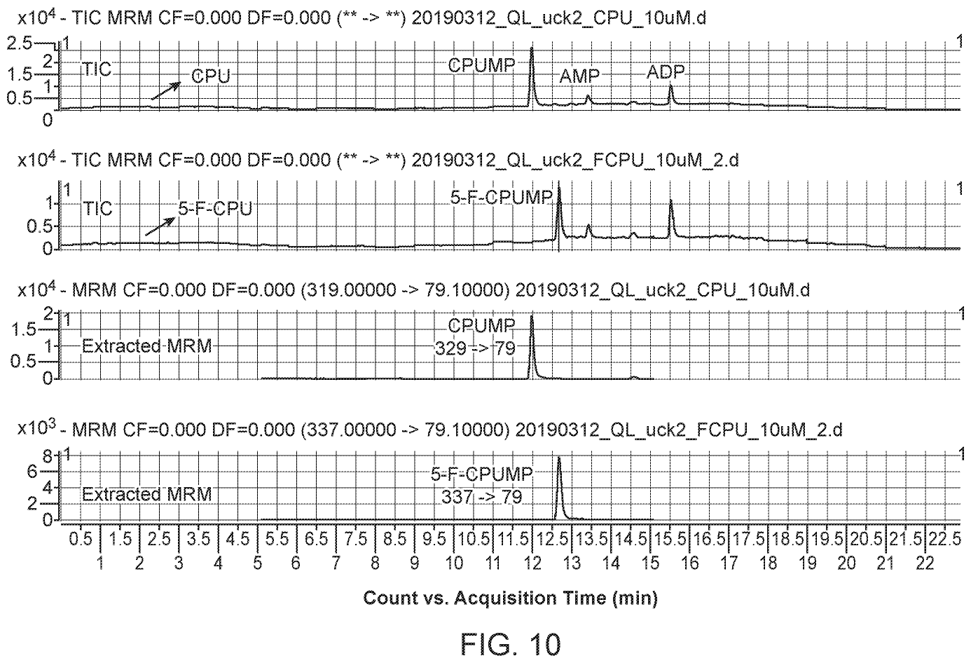

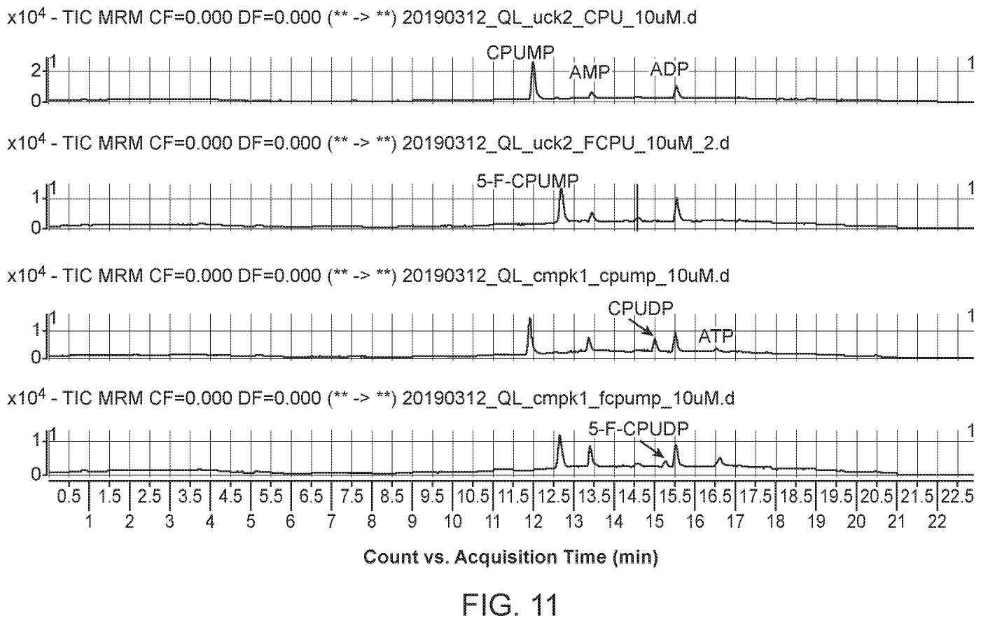

Given that CPU and 5-F-CPU could be phosphorylated by UCK2, we sought to establish whether the corresponding monophosphates were substrates or inhibitors of human CMPK1. For this purpose, human CMPK1 was expressed in E. coli and purified. We then employed recombinant UCK2 to synthesize CMP, CPU-MP and 5-F-CPU-MP and confirmed their identities by LC-MS/MS (FIG. 10). The results shown in FIG. 6 demonstrate that both CPU-MP and 5-F-CPU-MP are substrates of CMPK1, but that the former is a better substrate. Identities of their corresponding products, CPU-DP and 5-F-CPU-DP, were also confirmed by LC-MS/MS (FIG. 11).

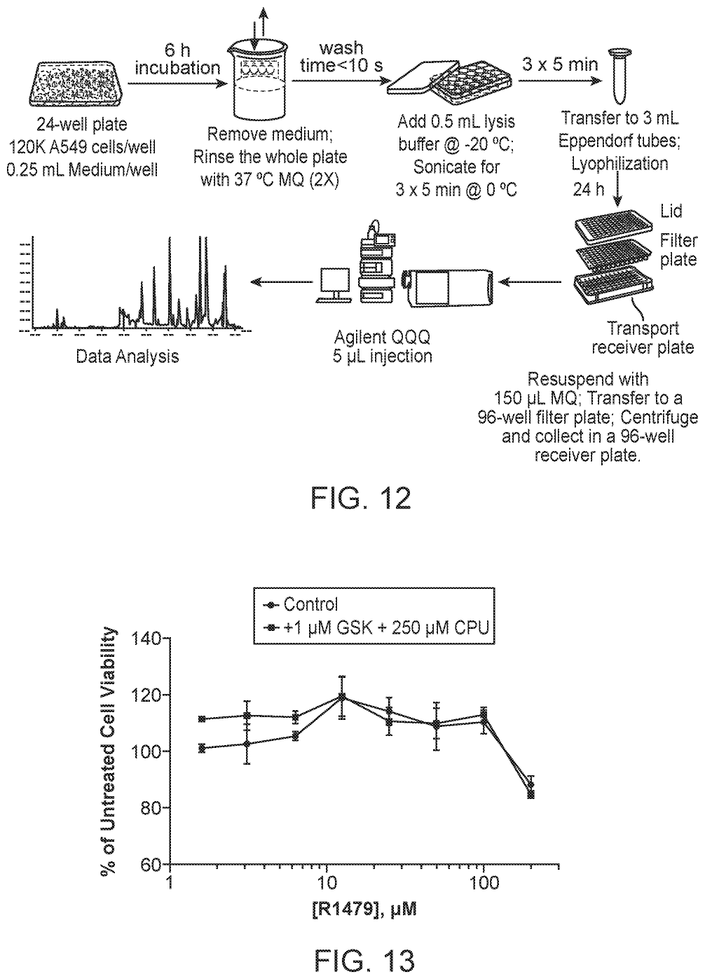

To understand the metabolic implications of the above bio-chemical findings, we developed a LC-MS/MS based assay that facilitated measurement of the effects of CPU and 5-F-CPU on intracellular pyrimidine nucleotide levels. To minimize the perturbative effects of sample quenching and analysis on the physiological concentrations of these metabolites, we adapted an earlier protocol for growing cells on glass cover slips to facilitate rapid washing (FIG. 12). LC-MS of nucleotides was performed using a dynamic multiple reaction monitoring (dMRM) method. Addition of micromolar concentrations of medronic acid into the mobile phase remarkedly increased the sensitivity for detecting di- and tri-nucleotides by this method.

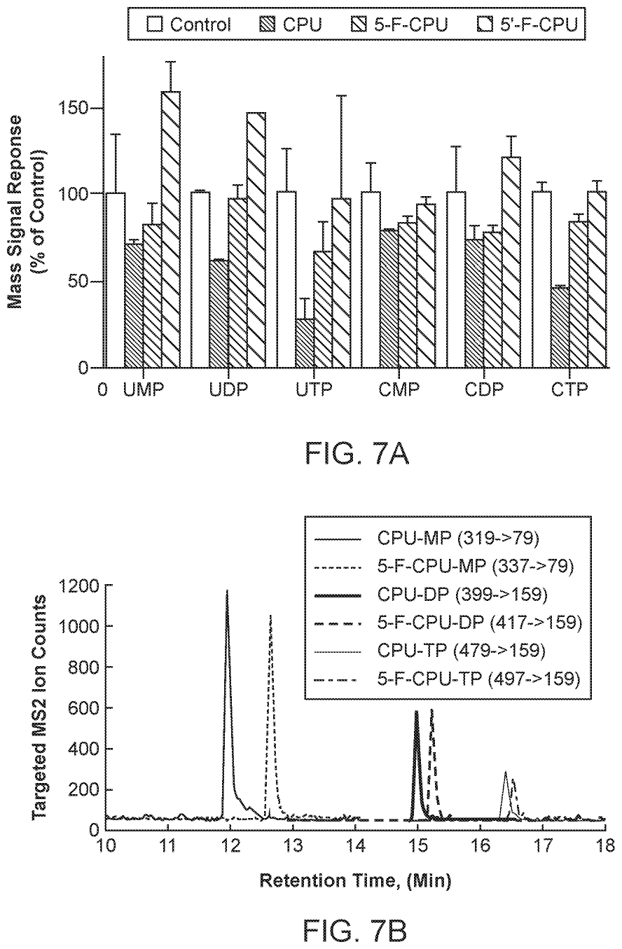

With optimized sampling and detection method in hand, pyrimidine nucleotide levels were measured in cells cultured with either 250 μM CPU, 5-F-CPU or 5′-F-CPU in combination with 1 μM GSK983 (FIG. 7A). Notwithstanding its UCK2 inhibitory activity, 5′-F-CPU did not deplete intracellular uridine and cytidine nucleotide levels. In contrast, inclusion of CPU or 5-F-CPU led to 25-40% decrease in UMP, CMP, UDP, and CDP levels compared to cells treated with GSK983 alone. CPU depleted UTP and CTP concentrations more strongly than 5-F-CPU. Triphosphates of both CPU and 5-F-CPU could be detected by LC-MS, with CPU-TP being more abundant (FIG. 7B), suggesting that nucleoside diphosphate kinase, a mammalian enzyme known to have broad substrate scope, could convert CPU-DP and 5-F-CPU-DP into their corresponding nucleoside triphosphate analogs.

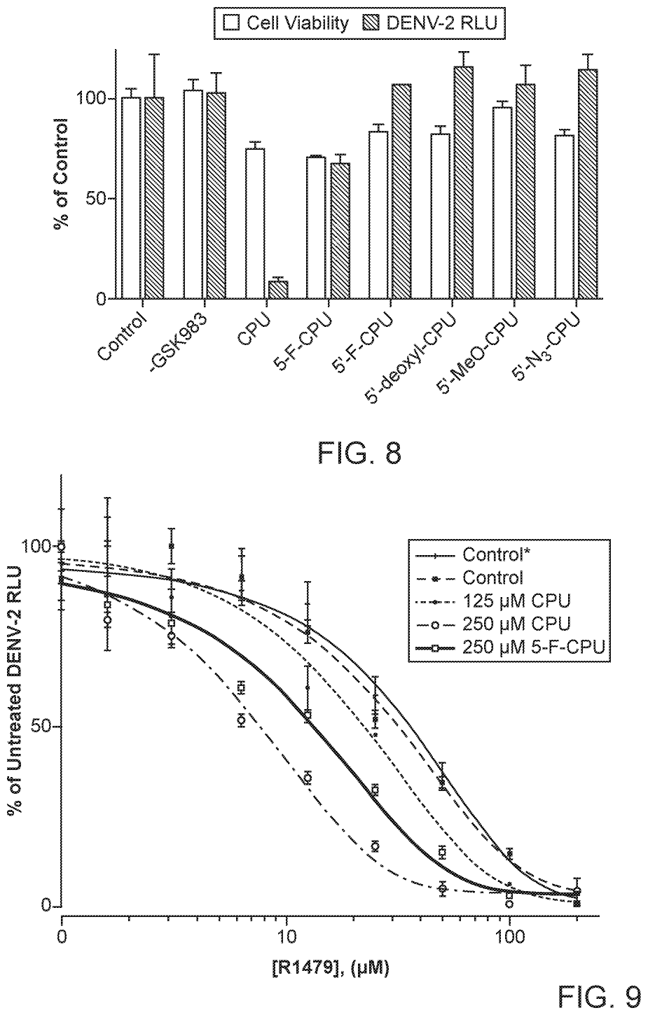

From above enzymological and metabolic data, we hypothesized that the combination of CPU and GSK983 would be more effective at inhibiting dengue virus replication than any other equivalent combinations. To test this prediction, we measured the antiviral and cytotoxic activities of selected CPU analogs at a concentration of 500 μM (FIG. 8). Antiviral activity was measured in a dengue virus replication assay, described earlier. In all assays, the culture medium was supplemented with 20 μM uridine to mimic plasma uridine concentrations. While neither GSK983 nor CPU were effective as single agents, the combination of both molecules suppressed dengue virus CPU) showed detectable antiviral activity. Consistent with the data shown in FIG. 2, CPU did not exhibit significant cytotoxic activity, nor did any of the CPU analogs tested.

The markedly higher antiviral activity of the CPU-GSK983 combination led us to hypothesize that, in addition to suppressing the intracellular pools of UTP and CTP, the antiviral effect of CPU could also, in part, be explained by the RdRp inhibitory effect of its triphosphate. Indeed, nucleoside analogs have been extensively studied for their ability to undergo phosphorylation and block RdRp activities. For example, the cytidine analogue 4t -azidocytidine (R1479) and its prodrug balapiravir (R1626) have been assessed for treating HCV, and later for dengue virus infections. However, both compounds failed in clinical practice against dengue viral infections due to limited efficacy and hematological toxicity. To test whether a combination therapy approach could improve the therapeutic index of R1479, we treated cells with R1479 in combination with GSK983 and CPU or 5-F-CPU (FIG. 9). As hypothesized, R1479 showed dose-dependent inhibition of dengue virus replication with an EC50 ˜35 μM. Inhibition of de novo pyrimidine biosynthesis via addition of GSK983 alone had negligible influence on the antiviral activity of R1479. In contrast, inhibition of both the de novo and salvage pathways 206 markedly enhanced the potency of R1479. In the presence of 250 μM CPU, the EC50 of R1479 was lowered to ˜8 μM. Notably, such 3-component treatment exhibited minimal impact on the cytotoxicity profile of R1479 (FIG. 13). Meanwhile, a similar effect was also observed when CPU was replaced with 5-F-CPU in this 3-component combination. The differences between the activities of CPU and 5-F-CPU in the presence (FIG. 9) versus absence (FIG. 8) of a bona fide RdRp inhibitor supports our hypothesis that CPU generates a relatively high intracellular ratio of CPU-TP:UTP due to its strong recognition by the enzymes responsible for pyrimidine salvage, whereas 5-F-CPU is considerably less effective at perturbing this ratio.

The flaviviruses, including dengue virus, are an important class of clinically-relevant viral pathogens with limited treatment options. In particular, dengue causes hundreds of millions of symptomatic infections annually yet lacks any antiviral treatment while the sole approved vaccine has limited use because of safety risks associated with vaccination of individuals not previously exposed to dengue. A series of high-throughput phenotypic cell-based screens have recently identified DHODH inhibition as a potent, broad-spectrum antiviral strategy in vitro. However, DHODH inhibitors lose antiviral activity upon extracellular uridine addition due presumably to pyrimidine salvage. While a prior study has explored combining a DHODH inhibitor with guanosine analogs ribavirin or INX-08189 in a non-uridine supplemented cell culture dengue model, the antiviral efficacy of a DHODH and a salvage inhibitor has not been previously reported despite interest. Herein, we found that a GSK983—CPU combination treatment effectively blocked dengue virus replication in the presence of physiological concentrations of extracellular uridine.

Synthesis and evaluation of a series of CPU analogs revealed that CPU and 5-F-CPU were substrates of UCK2, and the resulting monophosphates were substrates of CMPK1. The flux of CPU and 5-F-CPU to their triphosphate forms com- petitively depleted intracellular pools of pyrimidine NTPs and resulted in inhibition of RNA virus replication. At the same time, depletion of UTP and CTP led to a synergy between the CPU-GSK983 combination and RdRp inhibition.

CPU is known to be safe in mice, and does not show appreciable toxicity at a dose of 1 g/kg. While the safety profile of GSK983 remains unclear, other DHODH inhibitors like teriflunomide and brequinar have been extensively studied in humans, and are generally well-tolerated. Meanwhile, many RdRp inhibitors have been evaluated in clinical trials against dengue. Our results demonstrate that a combination strategy targeting both host pyrimidine biosynthesis and viral RdRp is useful as therapy against RNA viruses.

### Material and Methods

Unless otherwise noted, all reactions were performed under an argon atmosphere in flame- or oven-dried glassware. Reaction mixtures were stirred using Teflon-coated magnetic stirrer bars and monitored by thin layer silica gel chromatography (TLC) using 0.25 mm silica gel 60F plates with fluorescent indicator from Merck. Plates were visualized under UV or treated by KMnO4 stain with gentle heating. Products were purified on an AnaLogix IntelliFlash 280 Flash column chromatography system using the solvent gradients indicated. Anhydrous tetrahydrofuran (THF), dichloromethane (CH₂Cl₂), dimethylformamide (DMF), acetone, and dimethyl sufoxide (DMSO) were obtained from Acros Organics. Diethyl ether (Et₂O), ethyl acetate (EtOAc), hexanes, and methanol (MeOH) were from Fisher Scientific. DMSO used in bioassays and to prepare biological samples was from Fisher BioReagents. All other reagents were from commercial suppliers and were used as received without additional purification. Samples prepared for biological evaluation were purified via preparative HPLC in a water/acetonitrile (MeCN) gradient containing 0.1% (v/v) trifluoracetic acid (TFA) using an Agilent 1260 Infinity system equipped with an Agilent Prep-018 column (21.2×250 mm).

NMR spectra were measured on a Varian INOVA 500 (¹H at 500 MHz, ¹³C at 125 MHz), a Varian 400 (¹H at 400 MHz, ¹³C at 100 MHz), or a Varian INOVA 600 MHz (¹H at 500 MHz, ¹³C at 150 MHz) magnetic resonance spectrometer, as noted. ¹H chemical shifts were reported relative to the residual solvent peak (CDCl₃=7.26 ppm; MeOD=3.31 ppm) (1) as follows: chemical shift (δ) [multiplicity (s=singlet, brs=broad singlet, d=doublet, t=triplet, q=quartet, dd=doublet of doublet, m=multiplet), coupling constant(s) in Hz, integration]. ¹³C chemical shifts were reported relative to the residual deuterated solvent ¹³C signals (CDCl₃=77.16 ppm, MeOD=49.00 ppm) (1) and rounded to one decimal places. Infrared spectra were recorded on a Nicolet iS50 FT/IR Spectrometer at the Stanford Nano Share Facilities and were reported in wavenumbers (cm⁻¹). Optical rotation data were obtained using a JASCO DIP were reported as [α]_{D}²⁰ (c=grams/100 mL, solvent), where D indicates the sodium D line (589 nm). High resolution mass spectra were obtained on an Agilent 6545 QT of mass spectrometer at the Metabolic Chemistry Analysis Center at Stanford University.

Experimental Procedures and Characterization Data

3-benzoyl-1-(3aS,4R,6aR)-6-(((tert-butyldiphenylsilyl)oxy)methyl)-2,2-dimethyl-3a,6a-dihydro-4H-cyclopenta[d][1,3]dioxol-4-yl)pyrimidine-2,4(1H,3H)-dione (3a)

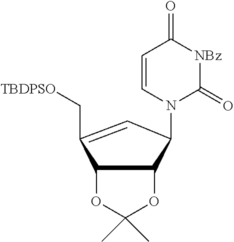

Compound 1 was prepared in 10 steps from D-ribose according to literature procedures. To a suspension of 1 (440 mg, 1.04 mmol, 1 equiv.), 2a (270 mg, 1.25 mmol, 1.2 equiv.), and PPh₃ (410 mg, 1.56 mmol, 1.5 equiv.) in THF (12 mL) at 0° C. was added DEAD solution in toluene (720 μL, 1.5 equiv., 40 w % in toluene). After the addition of DEAD, the reaction mixture turned from a white suspension to a yellow solution, which was slowly warmed up to room temperature and stirred overnight. The reaction mixture was then concentrated, loaded onto a 12 g SiO₂flash cartridge, and purified with a linear gradient of 20-40% EtOAc in hexanes to afford 3a (446 mg, 0.72 mmol, 69%) as white powder. [α]_{D}²⁰—17.0 (c 1.3, MeOH); IR (film, cm^{—1}) ;3071, 2930, 2857, 1748, 1705, 1667, 1441, 1429, 1372, 1234, 1112, 9055, 732, 704. ¹H NMR (400 MHz, CDCl₃) δ7.95 (d, J=7.4 Hz, 1H), 7.71-7.61 (m, 6H), 7.53-7.47 (m, 2H), 7.45-7.42 (m, 2H), 7.41-7.34 (m, 4H), 6.91 (d, J=8.1 Hz, 1H), 5.77 (d, J=8.1 Hz, 1H), 5.67 (s, 1H), 5.37 (s, 1H), 5.08 (d, J=5.8 Hz, 1H), 4.60 (d, J=5.8 Hz, 1H), 4.47 (d, J=16.4 Hz, 1H), 4.41 (d, J=16.7 Hz, 1H), 1.34 (s, 3H), 1.29 (s, 3H), 1.10 (s, 9H). ¹³C NMR (100 MHz, CDCl₃) δ168.8, 162.3, 153.6, 149.8, 141.1, 135.6, 135.6, 135.3, 133.3, 133.1, 131.6, 130.7, 130.1, 129.3, 128.0, 128.0, 120.8, 112.9, 102.4, 84.6, 83.4, 68.4, 61.4, 27.3, 27.0, 25.9, 19.4. HRMS (ESI) m/z 623.2574 [(M+H)⁺; calcd for C₃₆H₃₉N₂O₆Si⁺: 623.2572].

1-((1R,4R,5S)-4,5-dihydroxy-3-(hydroxymethyl)cyclopent-2-en-1-yl)pyrimidine-2,4(1H,3H)-dione (4a)

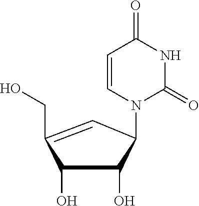

To compound 3a (30 mg, 0.048 mmol) was added 0.5 mL 7N NH₃ solution in methanol. After 1 h, the reaction solvent was removed under positive N₂ atmosphere and the resulting solid was further dried under high vacuum. The reaction crude was then treated with 30 μL HCl in 300 μL THF and stirred overnight. Excess NaHCO₃ was added to neutralize the reaction, followed by the addition of 1 mL MeOH. The resulting suspension was filtered through a short Celite pad, rinsed with MeOH, and concentrated. The resulting crude was then resuspended with 1 mL H₂O and subjected to HPLC purification with a linear gradient of 5-20% MeCN (0.1% TFA) in H₂O (0.1% TFA) on a prep C18 column (Agilent 10 prep-C18 250×21.1 mm). Fractions containing desired products was then combined and lyophilized to afford the final product 4a (4.6 mg, 0.019 mmol, 40%) as white powder. [α]_{D}²⁰—62.8 (c 3.2, MeOH); IR (film, cm⁻¹): 3349 (br), 1667, 1465, 1390, 1258, 1202, 1114. ¹H NMR (500 MHz, MeOD) δ7.42 (d, J=7.9 Hz, 1H), 5.71 (s, 1H), 5.49 (s, 1H), 4.90 (s, 1H), 4.52 (d, J=5.9, 1.2 Hz, 1H), 4.33 — 4.19 (m, 2H), 4.04 (t, J=5.6 Hz, 1H). ¹³C NMR (125 MHz, MeOD) δ166.4, 153.1, 152.1, 143.6, 125.5, 102.8, 78.4, 74.0, 67.4, 60.3. HRMS (ESI) m/z 263.0644 [(M+Na)⁺; calcd for C₁₀H₁₂N₂NaO₅⁺: 263.0638].

In a similar manner, compound 1 (360 mg, 0.85 mmol, 1 equiv.) was reacted with 2b (238 mg, 1.02 mmol, 1.2 equiv.) to afford intermediate 3b as white powder (288 mg, 0.45 mmol, 53%). Intermediate 3b (32 mg, 0.05 mmol) was then deprotected to afford 4b (7.7 mg, 0.03 mmol, 60%) as white powder.

3-benzoyl-1-((3aS,4R,6aR)-6-(((tert-butyldiphenylsilyl)oxy)methyl)-2,2-dimethyl-3a,6a-dihydro-4H-cyclopenta[d][1,3]dioxol-4-yl)-5-fluoropyrimidine-2,4(1H,3H)-dione (3b)

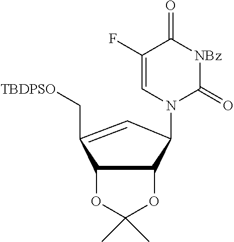

[α]_{D}²⁰—12.9 (c 1.4, CHCl₃); IR (film, cm⁻¹): 3072, 2932, 2857, 1753, 1712, 1665, 1662, 1448, 1373, 1234, 1106, 1088, 732, 702, 687. ¹H NMR (400 MHz, CDCl₃) δ7.97-7.92 (m, 2H), 7.73-7.63 (m, 5H), 7.55-7.49 (m, 2H), 7.48-7.42 (m, 2H), 7.42-7.35 (m, 4H), 7.06 (d, J=5.8 Hz, 1H), 5.68 (brs, 1H), 5.41 (brs, 1H), 5.08 (d, J=5.8 Hz, 1H), 4.59 (dd, J=12.1, 6.4 Hz, 1H), 4.54-4.35 (m, 2H), 1.34 (s, 3H), 1.29 (s, 3H), 1.11 (s, 9H). ¹³C NMR (100 MHz, CDCl₃) δ167.3, 154.3, 148.3, 141.4, 139.0, 135.6, 135.6, 133.2, 133.0, 131.2, 130.8, 130.2, 130.2, 129.4, 129.1, 128.0, 125.5, 125.1, 120.4, 113.0, 84.6, 83.2, 68.3, 61.4, 27.3, 27.0, 25.9, 19.4. ¹⁹F NMR (376 MHz, CDCl₃) δ−163.44 (d, J=5.7 Hz). HRMS (ESI) m/z 663.2323 [(M+Na)⁺; calcd for C₃₆H₃₇FN₂O₆SiNa⁺:663.2297].

1-((1R,4R,5S)-4,5-dihydroxy-3-(hydroxymethyl)cyclopent-2-en-1-yl)-5-fluoropyrimidine-2,4(1H,3H)-dione (4b)

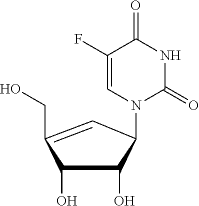

[α]_{D}²⁰—87.8 (c 0.45, MeOH); IR (film, cm⁻¹): 3375 (br), 1696, 1660, 1386, 1242, 1115, 1011. ¹H NMR (400 MHz, MeOD) δ7.60 (d, J=6.6 Hz, 1H), 5.68 (q, J=1.8 Hz, 1H), 5.49 (brs, 1H), 4.52 (d, J=5.6 Hz, 1H), 4.26 (d, J=2.2 Hz, 2H), 4.03 (t, J=5.5 Hz, 1H). ¹³C NMR (100 MHz, MeOD) δ159.54 (d, J=26.1 Hz), 152.64, 151.74, 142.07 (d, J=233.0 Hz), 127.39 (d, J=33.7 Hz), 125.24, 78.17, 73.94, 67.73, 60.26. ¹⁹F NMR (376 MHz, MeOD) δ−168.53 (dd, J=6.8, 1.7 Hz). HRMS (ESI) m/z 259.0722 [(M+H)⁺; calcd for C₁₀H₁₂FN₂O₅⁺: 259.0725].

In a similar manner, compound 1 (245 mg, 0.58 mmol, 1 equiv.) was reacted with 2c (238 mg, 0.70 mmol, 1.2 equiv.) to afford intermediate 3c (160 mg, 0.21 mmol, 36%) as white powder. Intermediate 3c (40 mg, 0.053 mmol) was then deprotected to afford 4c (9.6 mg, 0.026 mmol, 49%) as white powder.

3-benzoyl-1-((3aS,4R,6aR)-6-(((tert-butyldiphenylsilyl)oxy)methyl)-2,2-dimethyl-3a,6a-dihydro-4H-cyclopenta[d][1,3]dioxol-4-yl)-5-iodopyrimidine-2,4(1H,3H)-dione (3c)

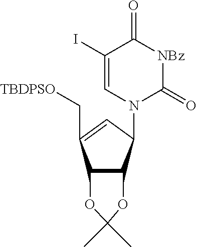

[α]_{D}²⁰—45.1 (c 1.3, CHCl₃); IR (film, cm⁻¹): 3071, 2932, 2857, 1749, 1703, 1666, 1608, 1420, 1233, 1112, 703. ¹H NMR (400 MHz, CDCl₃) δ7.93-7.90 (m, 1H), 7.72-7.63 (m, 5H), 7.54-7.48 (m, 3H), 7.45-7.37 (m, 6H), 5.72 (brs, 1H), 5.37 (s, 1H), 5.13 (d, J=5.8 Hz, 1H), 4.62 (d, J=5.8 Hz, 1H), 4.51-4.34 (m, 2H), 1.34 (s, 3H), 1.29 (s, 3H), 1.09 (s, 9H). ¹³C NMR (100 MHz, CDCl₃) δ167.8, 159.1, 154.1, 149.5, 145.8, 135.6, 135.4, 133.1, 133.1, 131.1, 130.7, 130.1, 129.4, 128.0, 128.0, 120.6, 113.0, 84.6, 83.4, 69.2, 68.1, 61.4, 27.3, 27.0, 25.9, 19.5. HRMS (ESI) m/z 749.1538 [(M+H)⁺; calcd for C₃₆H₃₈IN₂O₆Si⁺:749.1538].

1-((1R,4R,5S)-4,5-dihydroxy-3-(hydroxymethyl)cyclopent-2-en-1-yl)-5-iodopyrimidine-2,4(1H,3H)-dione (4c).

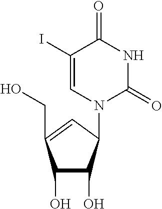

[α]_{D}²⁰—104.0 (c 0.6, MeOH); IR (film, cm⁻¹): 3370 (br), 1682, 1608, 1424, 1260, 1111. ¹H NMR (400 MHz, MeOD) δ7.78 (s, 1H), 5.70 (q, J=1.8 Hz, 1H), 5.47 (brs, 1H), 4.52 (d, J=5.5 Hz, 1H), 4.33-4.20 (m, J=2.1 Hz, 2H), 4.04 (t, J=5.7 Hz, 1H). ¹³C NMR (100 MHz, MeOD) δ163.0, 152.8, 152.4, 148.0, 125.5, 78.6, 73.9, 68.6, 67.9, 60.0. HRMS (ESI) m/z 366.9782 [(M+H)⁺; calcd for C₁₀H₁₂IN₂O₅⁺: 366.9785].

In a similar manner, compound 1 (113 mg, 0.27 mmol, 1 equiv.) was reacted with 2d (74 mg, 0.32 mmol, 1.2 equiv.) to afford intermediate 3d as white powder (100 mg, 0.16 mmol, 59%). Intermediate 3d (30 mg, 0.047 mmol) was then deprotected to afford 4d (4.3 mg, 0.017 mmol, 36%) as white powder.

3-benzoyl-1-((3aS,4R,6aR)-6-(((tert-butyldiphenylsilyl)oxy)methyl)-2,2-dimethyl-3a,6a-dihydro-4H-cyclopenta[d][1,3]dioxol4-yl)-5-methylpyrimidine-2,4(1H,3H)-dione (3d)

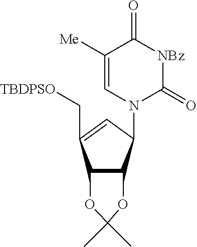

[α]_{D}²⁰—27.8 (c 1.2, CHCl₃); IR (film, cm⁻¹): 3071, 2932, 2857, 1748, 1699, 1656, 1429, 1371, 1235, 1111, 732, 702. ¹H NMR (500 MHz, CDCl3) δ7.97-7.89 (m, 2H), 7.71-7.61 (m, 5H), 7.53-7.46 (m, 3H), 7.45-7.42 (m, 1H), 7.40-7.35 (m, 4H), 6.90 (brs, 1H), 5.73 (s, 1H), 5.37 (s, 1H), 5.13 (d, J=5.8 Hz, 1H), 4.65 (dd, J=16.3, 4.7 Hz, 1H), 4.47 — 4.37 (m, 2H), 1.96 (s, 3H), 1.34 (s, 3H), 1.29 (s, 3H), 1.09 (s, 9H). ¹³C NMR (125 MHz, CDCl₃) δ169.1, 163.0, 152.9, 149.8, 137.2, 135.6, 135.1, 133.2, 133.1, 131.7, 130.6, 130.1, 129.3, 128.0, 121.3, 112.7, 111.0, 84.6, 83.5, 68.3, 61.4, 27.3, 27.0, 25.9, 19.5, 12.8. HRMS (ESI) m/z 637.2734 [(M+H)⁺; calcd for C₃₇H41N₂O₆Si⁺: 637.2728].

1-((1R,4R,5S)-4,5-dihydroxy-3-(hydroxymethyl)cyclopent-2-en-1-yl)-5-methylpyrimidine-2,4(1H,3H)-dione (4d)

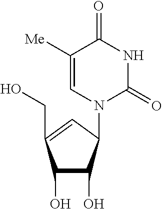

[α]_{D}²⁰—78.5 (c 0.38, MeOH); IR (film, cm⁻¹): 3371, 1683, 1476, 1261, 1205, 1114, 1016. ¹H NMR (400 MHz, MeOD) δ7.23 (d, J=1.2 Hz, 1H), 5.68 (q, J=1.8 Hz, 1H), 5.48 (brs, 1H), 4.52 (d, J=5.9 Hz, 1H), 4.26 (q, J=2.5 Hz, 2H), 4.04 (t, J=5.6 Hz, 1H), 1.87 (d, J=1.2 Hz, 3H). ¹³C NMR (100 MHz, MeOD) δ166.5, 153.3, 151.8, 139.2, 126.0, 111.8, 78.3, 74.0, 67.2, 60.3, 12.3. HRMS (ESI) m/z 277.0799 [(M+Na)⁺; calcd for C₁₁H₁₄N₂O₅Na⁺: 277.0795].

3-benzoyl-1-((3aS,4R,6aR)-6-(hydroxymethyl)-2,2-dimethyl-3a,6a-dihydro-4H-cyclopenta[d][1,3]dioxol-4-yl)pyrimidine-2,4(1H,3H)-dione (5)

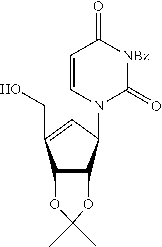

To a solution of 3a (300 mg, 0.48 mmol, 1 equiv.) in THF (5 mL) was added TBAF (580 μL, 0.58 mmol, 1.2 equiv., 1 M in THF) at 0° C. After 2 h, the reaction mixture was quenched with 5 mL of saturated NH₄OH solution and extracted with 5×5 mL EtOAc. The combined organic layers were dried over anhydrous Na₂SO₄, concentrated, loaded onto a 4 g SiO₂ flash cartridge, and purified with a linear gradient 80-95% EtOAc in hexanes to afford the free primary alcohol 5 (160 mg, 0.42 mmol, 87%) as white powder. [α]_{D}²⁰—4.9 (c 4.5, CHCl₃); IR (film, cm⁻¹): 3473 (br), 3088, 2988, 2933, 1746, 1702, 1665, 1443, 1374, 1239, 1179, 1239, 1059. ¹H NMR (500 MHz, CDCl₃) δ7.93 (d, J=8.4 Hz, 2H), 7.70-7.61 (m, 1H), 7.50 (dd, J=8.2, 7.4 Hz, 2H), 7.16 (d, J=8.0 Hz, 1H), 5.81 (d, J=8.0 Hz, 1H), 5.64 (s, 1H), 5.30 (s, 1H), 5.25 (d, J=5.8 Hz, 1H), 4.70 (d, J=5.8 Hz, 1H), 4.43 (d, J=15.9 Hz, 1H), 4.38 (d, J=15.9 Hz, 1H), 1.43 (s, 3H), 1.34 (s, 3H). ¹³C NMR (125 MHz, CDCl₃) δ168.8, 162.2, 152.5, 149.7, 141.6, 135.3, 131.5, 130.7, 129.3, 121.7, 113.0, 102.5, 84.3, 84.0, 69.2, 60.2, 27.3, 25.8. HRMS (ESI) m/z 385.1391 [(M+H), calcd for C₂₀H₁₂N₂O₆⁺: 385.1394].

1-((1R,4R,5S)-4,5-dihydroxy-3-(methoxymethyl)cyclopent-2-en-1-yl)pyrimidine-2,4(1H,3H)-dione (6)

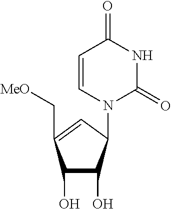

To a solution of 5 (20 mg, 0.05 mmol, 1 equiv.) in dry acetone (0.5 mL) was added Ag₂O (23 mg, 0.1 mmol, 2 equiv.) and Mel (31 μL, 0.5 mmol, 10 equiv.). The reaction mixture was stirred at room temperature for 24 h, filtered through Celite pad, and concentrated in vacuo to afford a residue oil 17 mg. In a similar manner as the synthesis of 3a from 4a, the crude was treated with 0.5 mL 7N NH₃ in methanol, followed by 25 μL HCl in 250 THF to furnish the analog 6 (2.5 mg, 0.01 mmol, 20% for three steps). [α]_{D}²⁰—64.7 (c 0.25, MeOH); IR (film, cm⁻¹) ; 3369 (br), 2921, 2851, 1682, 1469, 1410, 1262, 1204, 1096. ¹H NMR (600 MHz, MeOD) δ7.40 (d, J=8.0 Hz, 1H), 5.72 (dd, J=3.8, 1.7 Hz, 1H), 5.69 (d, J=8.0 Hz, 1H), 5.51 — 5.43 (m, 1H), 4.50 (d, J=5.8 Hz, 1H), 4.14 (ddd, J=14.1, 2.5, 2.4 Hz, 1H), 4.08 (ddd, J=14.2, 2.2, 1.8 Hz, 1H), 4.05 (dd, J=5.7, 5.5 Hz, 1H), 3.40 (s, 3H). ¹³C NMR (100 MHz, MeOD) δ166.4, 153.0, 148.8, 143.7, 127.5, 102.8, 78.1, 74.0, 70.3, 67.6, 59.0. HRMS (ESI) m/z 277.0799 [(M+Na)⁺; calcd for C₁₁H₁₄N₂O₅Na⁺: 277.0795].

3-benzoyl-1-((3aS,4R,6aR)-6-(fluoromethyl)-2,2-dimethyl-3a,6a-dihydro-4H-cyclopenta[d][1,3]dioxol-4-yl)pyrimidine-2,4(1H,3H)-dione (S1)

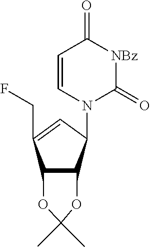

To alcohol 5 (50 mg, 0.13 mmol, 1 equiv.) in 1 mL CH₂Cl₂ at −78° C. was added a stock solution of DAST (0.15 mmol, 1.2 equiv., 200 μL, prepared by dilute 100 μL of DAST with CH₂Cl₂ to 1 mL). The reaction mixture was warmed up to room temperature and stirred overnight before quenching with 1 mL saturated NaHCO₃. The resulting mixture was then extracted with 3×3 mL EtOAc. The combined organic layers were dried over anhydrous Na₂SO₄, concentrated, loaded onto a 4 g SiO₂ flash cartridge, and purified with a linear gradient 20-50% EtOAc in hexanes to afford the intermediate S1 (27 mg, 0.070 mmol, 54%) as white powder. [α]_{D}²⁰—12.8 (c 2.4, CHCl₃); IR (film, cm⁻¹): 3100, 2989, 2937, 1746, 1704, 1667, 1599, 1441, 1374, 1239, 1179, 1060,989. ¹H NMR (600 MHz, CDCl3) δ7.93 (d, J=8.1 Hz, 2H), 7.66 (t, J=7.5 Hz, 1H), 7.51 (dd, J=7.91, 7.13 Hz, 2H), 7.13 (d, J=8.1 Hz, 1H), 5.83 (d, J=8.1 Hz, 1H), 5.72 (s, 1H), 5.32 (s, 1H), 5.26 (d, J=5.9 Hz, 1H), 5.14 (s, 1H), 5.07 (s, 1H), 4.73 (d, J=5.7 Hz, 1H), 1.43 (s, 3H), 1.34 (s, 3H). ¹³C NMR (125 MHz, CDCl₃) δ168.7, 162.2, 149.6, 141.5, 135.4, 131.5, 130.7, 129.4, 123.2, 113.2, 102.7, 95.7, 84.1, 83.2, 80.5 (d, J=149 Hz), 78.8, 77.4, 77.2, 76.9, 69.2, 27.3, 25.8. ¹⁹F NMR (376 MHz, CDCl₃) δ−224.4 (t, J=46.5). HRMS (ESI) m/z 387.1366 [(M+H)⁺; calcd for C₂₀H₂₀FN₂O₅⁺: 387.1351].

1-((1R,4R,5S)-3-(fluoromethyl)-4,5-dihydroxycyclopent-2-en-1-yl)pyrimidine-2,4(1H,3H)-dione (7)

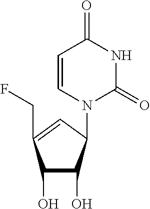

In a similar manner as the synthesis of 4a from 3a, compound S1 (13 mg, 0.034 mmol, 1 equiv.) was treated with 0.5 mL 7N NH₃ in methanol, followed by 25 μL HCl in 250 μL THF to furnish the analog 7 (4.4 mg, 0.018 mmol, 53% for two steps) as white powder. [α]_{D}²⁰—86.0 (c 0.22, MeOH); IR (film, cm⁻¹): 3365 (br), 2920, 2852, 1675, 1466, 1389, 1265, 1203, 1115. ¹H NMR (600 MHz, MeOD) δ7.42 (d, J=7.9 Hz, 1H), 5.85 (brs, 1H), 5.70 (d, J=8.0 Hz, 1H), 5.48 (brs, 1H), 5.15-4.99 (m, 2H), 4.57 (d, J=6.0 Hz, 1H), 4.10 (dd, J=6.7, 6.0 Hz, 1H). ¹³C NMR (150 MHz, MeOD) δ166.2, 152.8, 147.2, 143.6, 128.2, 102.8, 80.5 (d, J=149 Hz), 77.6, 73.1, 67.4. ¹⁹F NMR (376 MHz, MeOD) δ−224.9 (t, J=46.8). HRMS (ESI) m/z 265.0592 [(M+Na)⁺; calcd for C₁₀H₁₁FN₂O₄Na⁺: 265.0595].

((3aS,4R,6aR)-4-(3-benzoyl-2,4-dioxo-3,4-dihydropyrim idin-1(2 H)-yl)-2,2-dimethyl-3a,6a-dihydro-4H-cyclopenta[d][1,3]dioxol-6-yl)methyl methanesulfonate (8)

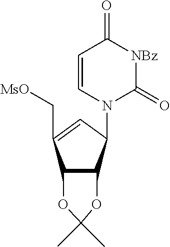

To a solution of 5 (60 mg, 0.16 mmol, 1 equiv.) in anhydrous CH₂Cl₂ (2 mL) was added Et₃N (44 μL, 0.32 mmol, 2 equiv.) at 0° C., followed by the dropwise addition of 0.2 mL MsCl stock solution (0.26 mmol, 1.6 equiv.; Stock solution was prepared by diluting 0.1 mL MsCl with CH₂Cl₂ to 1 mL). After 20 min at 0° C., the reaction mixture was quenched with 2 mL saturated aqueous solution of NH₄Cl, extracted with 3×3 mL CH₂Cl₂, dried over anhydrous Na₂SO₄, concentrated, loaded on a 4 g SiO₂ flash cartridge, and purified with a linear gradient 50-95% EtOAc in hexanes to afford the compound 8 (53 mg, 0.11 mmol, 72%) as pale yellow oil. [α]_{D}²⁰—35.3 (c 2.8, CHCl₃); IR (film, cm⁻¹): 2989, 2937, 1745, 1702, 1661, 1597, 1440, 1354, 1235, 1174, 1087, 959, 906, 729. ¹H NMR (500 MHz, CDCl₃) δ7.92 (d, J=7.2 Hz, 2H), 7.66 (dd, J=8.0, 7.7 Hz, 1H), 7.50 (d, J=7.8 Hz, 2H), 7.19 (d, J=8.1 Hz, 1H), 5.83 — 5.67 (m, 2H), 5.30 (d, J=4.8 Hz, 2H), 4.98 (d, J=14.0 Hz, 1H), 4.83 (d, J=14.0 Hz, 1H), 4.71 (d, J=5.8 Hz, 1H), 3.04 (s, 3H), 1.41 (s, 3H), 1.33 (s, 3H). ¹³C NMR (125 MHz, CDCl₃) δ168.7, 162.1, 149.6, 146.3, 141.8, 135.4, 131.4, 130.6, 129.4, 125.9, 113.1, 102.7, 83.8, 83.1, 77.4, 77.2, 76.9, 68.7, 65.0, 38.1, 27.3, 25.7. HRMS (ESI) m/z 463.1185 [(M+H)⁺; calcd for C₂₁H₂₃N₂O₈S⁺: 463.1170].

1-((3aS,4R,6aR)-6-(azidomethyl)-2,2-dimethyl-3a,6a-dihydro-4H-cyclopenta[d][1,3]dioxol-4-yl)pyrimidine-2,4(1H,3H)-dione (S2)

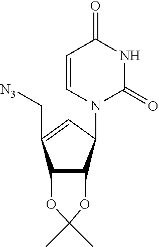

To the mesylate intermediate 8 (26 mg, 0.056 mmol, 1 equiv.) was added DMF (3 mL) and NaN₃ (80 mg, 1.23 mmol, 22 equiv.). The resulting suspension was heated to 100° C. and stirred for 18 hours, followed by the addition of 3 mL saturated solution of NH₄Cl. The organic layer was extracted with 3×5 Et₂O, washed with brine, and concentrated under reduced pressure. The crude material was purified through a 4 g SiO₂ flash cartridge with a linear gradient 40-95% EtOAc in hexanes to afford the azide intermediate S2 (14 mg, 0.046 mmol, 82%) as brown oil. [α]_{D}²⁰—34.0 (c0.7, CHCl₃); IR (film, cm⁻¹): 3197, 3059, 2988, 2929, 2102, 1687, 1456, 1380, 1243, 1082, 1058. ¹H NMR (500 MHz, CDCl₃) δ8.78 (brs, 1H), 7.02 (d, J=8.0 Hz, 1H), 5.71 (d, J=8.0 Hz, 1H), 5.64 (brs, 1H), 5.31 (brs, 1H), 5.22 (d, J=5.7 Hz, 1H), 4.65 (d, J=5.8 Hz, 1H), 4.13 (d, J=16.0 Hz, 1H), 4.04 (d, J=16.0 Hz, 1H), 1.44 (s, 3H), 1.35 (s, 3H). ¹³C NMR (125 MHz, CDCl₃) δ162.8, 150.5, 150.1, 141.2, 122.1, 112.6, 102.3, 86.2, 84.4, 68.0, 27.4, 26.0, 14.6. HRMS (ESI) m/z 306.1195 [(M+H)⁺; calcd for C₁₃H₁₆N₅O₄⁺: 306.1197].

1-((1 R,4R,5S)-3-(azidomethyl)-4,5-dihydroxycyclopent-2-en-1-yl)pyrimidine-2,4(1H,3H)-dione (9)

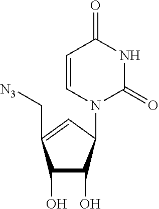

To intermediate S2 (6 mg, 0.023 mmol, 1 equiv.) was added THF (250 μL) and concentrated HCl (25 μL). The reaction mixture was stirred at room temperature for 5 h. An excess amount of NaHCO₃ was added to neutralize the reaction, followed by the addition of 1 mL MeOH. The resulting suspension was then filtered through a short Celite pad, rinsed with 2×2 MeOH, and concentrated. The resulting crude was resuspended with 1 mL H₂O and subjected to HPLC purification with a linear gradient of 5-20% MeCN (0.1% TFA) in H₂O (0.01% TFA) on a prep C18 column (Agilent 10 prep-C18 250×21.1 mm). Fractions containing desired products were then combined and lyophilized to afford the final product 9 (3.6 mg, 0.014 mmol, 59%) as white powder. [α]_{D}²⁰—50.0 (c 0.36, MeOH); IR (film, cm⁻¹): 3358 (br), 2921, 2104, 1683, 1467, 1393, 1261, 1205, 1116. ¹H NMR (400 MHz, MeOD) δ7.39 (d, J=8.0, 0.6 Hz, 1H), 5.81 (dd, J=3.2, 1.9 Hz, 1H), 5.70 (d, J=8.0 Hz, 1H), 5.52 — 5.40 (m, 1H), 4.52 (d, J=5.8 Hz, 1H), 4.14-3.97 (m, 3H). ¹³C NMR (100 MHz, MeOD) δ166.4, 153.0, 146.7, 143.7, 128.5, 102.9, 77.9, 74.5, 67.9, 50.4. HRMS (ESI) m/z 288.0697 [(M+Na)⁺; calcd for C₁₀H₁₁N₅O₄Na⁺: 288.0703].

((3aS,4R,6aR)-4-(3-benzoyl-2,4-dioxo-3,4-dihydropyrimidin-1(2H)-yl)-2,2-dimethyl-3a,6a-dihydro-4H-cyclopenta[d][1,3]dioxol-6-yl)methyl acetate (10)

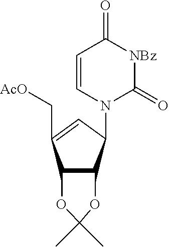

Triethylamine (66 uL, 0.48 mmol, 3 equiv.) and acetyl chloride (AcCl) (17 μL, 0.24 mmol, 1.5 equiv.) were sequentially added to a solution of 5 (60 mg, 0.16 mmol, 1 equiv.) in CH₂Cl₂ (2 mL) at 0° C. The reaction mixture was then warmed up to room temperature and stirred for another hour. A saturated solution of NH₄Cl (2 mL) was added. The organic layer was extracted with 3×3 CH₂Cl₂, washed with H₂O, and concentrated under reduced pressure. The crude material was purified through a 4 g SiO₂ flash cartridge with a linear gradient 65-95% EtOAc in hexanes to afford the intermediate 10 (56 mg, 0.13 mmol, 82%) as colorless oil. [α]_{D}²⁰—62.8 (c 1.3, CHCl₃); IR (film, cm⁻¹): 3080, 2988, 2935, 1742, 1703, 1663, 1599, 1440, 1372, 1236, 1179, 1236, 1058, 904, 731. ¹H NMR (400 MHz, CDCl₃) δ7.93 (d, J=7.6 Hz, 2H), 7.78-7.61 (m, 1H), 7.50 (dd, J=8.1, 7.7 Hz, 2H), 7.10 (d, J=8.1 Hz, 1H), 5.81 (d, J=8.1 Hz, 1H), 5.61 (brs, 1H), 5.34 (brs, 1H), 5.23 (d, J=5.7 Hz, 1H), 4.79 (qt, J=15.0, 1.8 Hz, 2H), 4.67 (d, J=5.3 Hz, 1H), 2.12 (s, 3H), 1.42 (s, 3H), 1.34 (s, 3H). ¹³C NMR (100 MHz, CDCl₃) δ170.7, 168.7, 162.2, 149.7, 148.3, 141.2, 135.3, 131.5, 130.7, 129.3, 123.2, 113.1, 102.6, 84.1, 83.7, 68.6, 60.9, 27.3, 25.9, 20.9. HRMS (ESI) m/z 427.1508 [(M+H)⁺; calcd for C₂₂H₂₃N₂O₇⁺: 427.1500].

1-((1R,4R,5S)-4,5-dihydroxy-3-methylcyclopent-2-en-1-yl)pyrimidine-2,4(1H,3H)-dione (11)

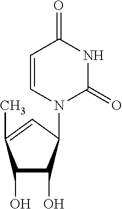

To a solution of the allylic acetate 10 (56 mg, 0.13 mmol, 1 equiv.) in 95% ethanol (4 ml) and cyclohexene (2 ml) was added 20 w % Pd(OH)₂ on carbon (20 mg, 1:3 catalyst substrate by weight). The resulting suspension was stirred under reflux overnight. The reaction mixture was then filtered, concentrated, and dried under high vacuum to afford 20 mg crude material as pale-yellow oil. In a similar manner as the preparation of 9, 6 mg of the crude product was treated with 25 μHCl in 250 μL THF to afford the final product (2.4 mg, 0.011 mmol, 28% for three steps) as white solid. [α]_{D}²⁰—78(c 0.24, MeOH); IR (film, cm⁻¹): 3364 (br), 2921, 2851, 1680, 1468, 1393, 1251, 1203, 1116. ¹H NMR (400 MHz, MeOD) δ7.39 (d, J=8.0 Hz, 1H), 5.67 (d, J=8.0 Hz, 1H), 5.52-5.35 (m, 2H), 4.37 (d, J=5.8 Hz, 1H), 3.99 (dd, J=5.9, 5.4 Hz, 1H), 1.88 (s, 3H). ¹³C NMR (100 MHz, MeOH) δ166.4, 153.1, 148.9, 143.5, 125.6, 102.7, 78.2, 77.2, 68.1, 15.1. HRMS (ESI) m/z 247.0685 [(M+Na)⁺; calcd for C₁₀H₁₂N₂O₄Na: 247.0689].

Chemicals and Reagents: For spectrophotometric enzyme assays, reagents included lactate dehydrogenase (EMD Millipore, porcine heart), pyruvate kinase (Sigma, rabbit muscle type III), NADH disodium salt (ChemCruz), phosphoenolpyruvate monopotassium salt (Sigma, 97%), ATP disodium trihydrate (VMR Life Science, ultrapure) cytidine 5′-monophospahte disodium salt (Sigma, ≥99%), uridine (Sigma, ≥99%), and cytidine (Sigma, 99%),

Cell culture: All cell lines were maintained in a humidified incubator (37° C., 5% CO₂). A549 cells were obtained from ATCC and cultured in DMEM supplemented with 10% FBS, penicillin/streptomycin, and L-glutamine. Upon reaching 50-75% confluence, A549 cells were detached from the growth surface using a trypsin/EDTA solution prior to analysis. Cells were maintained in logarithmic growth during all biological assays.

DENV-Luc reporter virus generation: The design of the pDENV-Luc infectious clone derived from dengue serotype 2 (DENV-2) strain 16681 was described in detail by Marceau et al (Marceau et al., 2016). The plasmid was linearized with Xbal and in vitro transcribed into the genomic RNA of DENV-Luc virus using the T7 Megascript Kit (Ambion) in the presence of m7G(5′)ppp(5′)G RNA Cap Structure Analog (NEB). 5 μg DENV-Luc RNA was electroporated into 2×10⁶ Vero cells. The transfected cells were resuspended with Dulbecco's modified Eagle's medium (DMEM; Invitrogen) with 10% FBS and 100 U/ml penicillin-streptomycin and transferred into a T-175 flask incubated at 37° C. with 5% CO₂. Supernatants were collected and replenished with fresh medium every 24 h from day 17 to 24 post-transfection, pooled together, clarified by centrifugation and stored in aliquots at −80° C. The amount of infectious DENV-Luc virus in the stock was titrated using the TCID50 assay and calculated by the Spearman & Kärber algorithm as described previously (Hierholzer and Killington, 1996).

Pilot Antiviral Assays (for FIG. 2): A549 cells were plated overnight at 20,000 cells/well in 24-well plates in complete DMEM and incubated for 24 h at 37° C. Cell were then treated with pyrimidine de novo synthesis and/or salvage inhibitors in DMEM supplemented with 20 μM uridine. Four hours after drug addition, cells were infected with DENV-2 virus at a MOI=0.01. After 48 h, DENV-Luc replication was monitored by the production of Renilla luciferase, which was measured using the Renilla-Glo Luciferase Assay System (Promega) according to the specifications of the manufacturer. For the accompanying cell viability assay, A549 cells were seeded into 24-well plates at a density of 20,000 cells/well incubated for 24 h at 37° C. Cell were then treated with pyrimidine de novo synthesis and/or salvage inhibitors in DMEM supplemented with 20 μM uridine. Following 48h treatment, cells were harvested, and the density of viable cells was determined by flow cytometry (FSC/SSC) using a BD Accuri C6 Flow Cytometer.

Cloning, expression and purification of recombinant human UCK2 and CMPK1: Uridine cytidine kinase 2 (UCK2) was cloned into the pET-21a(+) vector using Ndel and Notl as restriction sites. This resulted in C-terminally 6×His-tagged UCK2. The resulting plasmid was transformed into E. coli BL21(DE3) cells by electroporation, recovered in SOC at 37° C. for 1 h and plated onto LB agar plates with kanamycin overnight at 37° C. A single, isolated colony was inoculated into 30 mL LB supplemented with 50 μg/mL kanamycin and grown at 37° C. with shaking for 15 h. The next day, these cells were used to inoculate 1 L autoclaved LB with 50 μg/mL kanamycin. The flask was shaken at 37° C. When an optical density (OD₆₀₀) of 0.7 was reached, protein expression was induced with 0.5 mM isopropyl β-D-1-thiogalactopyranoside (IPTG). The temperature was changed to 18° C. and flasks were shaken for an additional 15 h. The cell pellets were collected by spinning the media at 5000 rpm for 10 min. The pellets obtained were frozen by liquid nitrogen and stored in −80° C. for protein purification. Cell pellets were thawed and resuspended in lysis buffer containing Tris (40 mM, pH 7.5), NaCl (10 mM), imidazole (10 mM), DTT (1 mM). Cells were lysed by sonication and centrifuged at 25,000 g for 1 h. The supernatant was incubated with a slurry of Ni-NTA resin for 1 h at 4° C. and loaded onto a column. The nickel column wash buffer was Tris (40 mM, pH 7.5), NaCl (10 mM), DTT (1 mM) and each wash step contained increasing concentrations of imidazole (10, 50, or 250 mM). After SDS-PAGE confirmation of the protein fractions, protein was further purified using fast protein liquid chromatography (FPLC) using buffer A (50 mM Tris-HCl pH 8, 1 mM dithiothreitol (DTT), 10% glycerol) and elution buffer B (50 mM Tris-HCl pH 8, 1mM dithiothreitol (DTT), 10% glycerol with 500 mM NaCl) with changing gradient over a 20 minute from 2% to 100% Buffer B. FPLC eluents containing the protein was concentrated using Am icon centrifugal filter with 3K cut-off and the enzyme was stored in storage buffer (50 mM Tris-HCl pH 7.5, 10% glycerol). Human cytidine monophosphate kinase 1 (CMPK1) was purified as previously described (Deans et al., 2016).

In vitro enzyme activity assays with recombinant human UCK2: To continuously monitor reaction progress spectrophotometrically, ATP hydrolysis was coupled to NADH oxidation via pyruvate kinase (PK) and lactate dehydrogenase (LDH). Reactions were conducted at room temperature in 100 μL in 96-well plates (Greiner Bio-One, UV-Star, Half Area). Mixtures contained 20 mM HEPES (pH 7.2), 100 mM KCl, 2 mM MgCl₂, 300 μM ATP, 0-500 μM uridine, 0-500 μM CPU analogs, 10 nM UCK2, 1 mM phosphoenolpyruvate, 500 μM NADH and 20 units/mL of PK and LDH. Progress was monitored in the linear region using a Biotek Synergy HT and kinetic and inhibition constants were determined using GraphPad Prism 7 (GraphPad Software).

Enzymatic synthesis of CMP, CPU-MP, and 5-F-CPU-MP: Reactions were conducted at room temperature in 100 μL in Eppendorf tubes. Mixtures contained 20 mM HEPES (pH 7.2), 100 mM KCl, 2 mM MgCl₂, 2.5 mM ATP, 5 mM substrates (cytidine, CPU and 5-F-CPU), and 2 μM UCK2. After gently mixing for 24 h on a rocking shaker, the reaction mixtures were heated for 3 minutes at 95° C. to denature UCK2 enzyme. Formation of CPUMP and 5-F-CPUMP were confirmed by LC-MS/MS (FIG. 10).

DENV antiviral assays (For FIGS. 8 and 9): A549 cells were plated overnight at 5,000 cells/well in 96-well plates in complete DMEM and incubated for 24 h at 37° C. Cells were then treated with pyrimidine de novo synthesis inhibitors, and/or salvage inhibitors, and/or R1479 in DMEM supplemented with 20 μM uridine. Four hours after drug addition, cells were infected with DENV-2 virus at a MOI=0.1. After 72 h, DENV-Luc replication was monitored by the production of Renilla luciferase, which was measured using the Renilla-Glo Luciferase Assay System (Promega) according to the specifications of the manufacturer. For the accompanying cell viability assay, A549 cells were seeded at 5,000 cells/well in 96-well plates in complete DMEM and incubated for 24 h at 37° C. Cell were then treated with pyrimidine de novo synthesis inhibitors, and/or salvage inhibitors, and/or R1479 in DMEM supplemented with 20 μM uridine. Following 48 h treatment, cell viability was monitored by ATP levels, which was measured using the CellTiter-Glo Luciferase Assay System (Promega) according to the specifications of the manufacturer.

TABLE 1
Retention time and products monitored by LC-MS

| | | | | | | | Cell |
|---|---|---|---|---|---|---|---|
| Compound | | | Ret. Time | Delta | Frag | Collision | Accelerator |
| Name | Precursor Ion | Product Ion | (min) | Ret. Time | m-entor | Energy | Voltage |
| Uridine-5,6-d | 245.1 | 112 | 2.3 | 5 | 128 | 12 | 4 |
| UMP | 323 | 79.1 | 13 | 10 | 126 | 48 | 4 |
| UDP | 403 | 79 | 15.6 | 10 | 108 | 48 | 4 |
| UTP | 483 | 158.9 | 18 | 10 | 146 | 36 | 4 |
| CMP | 322 | 79 | 12 | 10 | 130 | 44 | 4 |
| CDP | 402.01 | 79 | 17 | 10 | 108 | 48 | 4 |
| CTP | 482 | 158.8 | 18.5 | 10 | 142 | 40 | 4 |
| CPU-MP | 319 | 79.1 | 10.1 | 10 | 126 | 48 | 4 |
| CPU-DP | 399 | 158.9 | 14 | 10 | 108 | 28 | 4 |
| CPU-TP | 479 | 159 | 16 | 10 | 146 | 36 | 4 |
| 5-F-CPU-MP | 337 | 79.1 | 10.1 | 10 | 126 | 48 | 4 |
| 5-F-CPU-DP | 417 | 158.9 | 14 | 10 | 108 | 28 | 4 |
| 5-F-CPU-TP | 497 | 159 | 16 | 10 | 146 | 36 | 4 |

1. A method of treating or preventing a virus infection, the method comprising administering to a subject:
(i) an effective dose of an inhibitor of dihydroorotate dehydrogenase (DHODH); and
(ii) an effective dose of a cyclopentenyl uracil (CPU) analog,
where the combined dose is effective to inhibit replication of a virus in a cell.

2. The method of claim 1, wherein the virus is an RNA virus.

3. The method of claim 2, further comprising administering the subject an effective dose of an inhibitor of RNA dependent RNA polymerase (RdRp).

4. The method of claim 1, wherein the CPU analog is selected from the group:

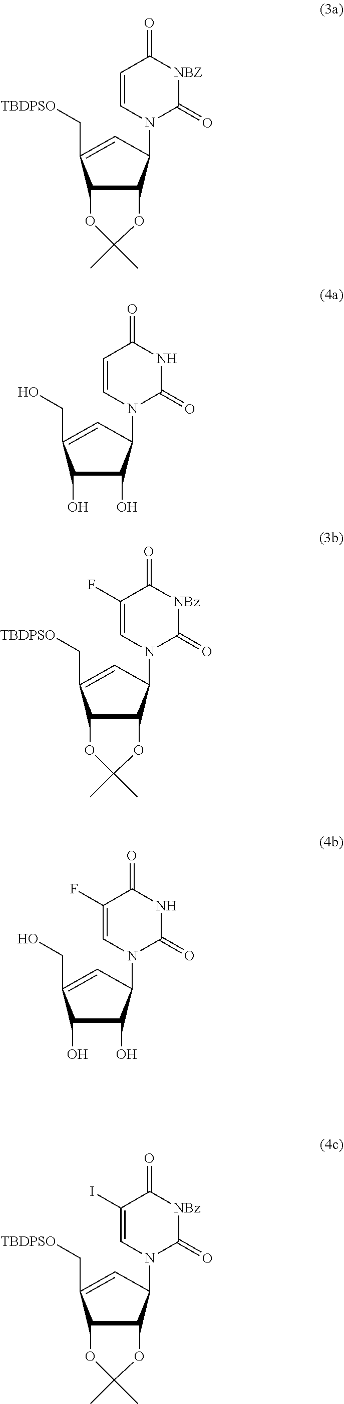

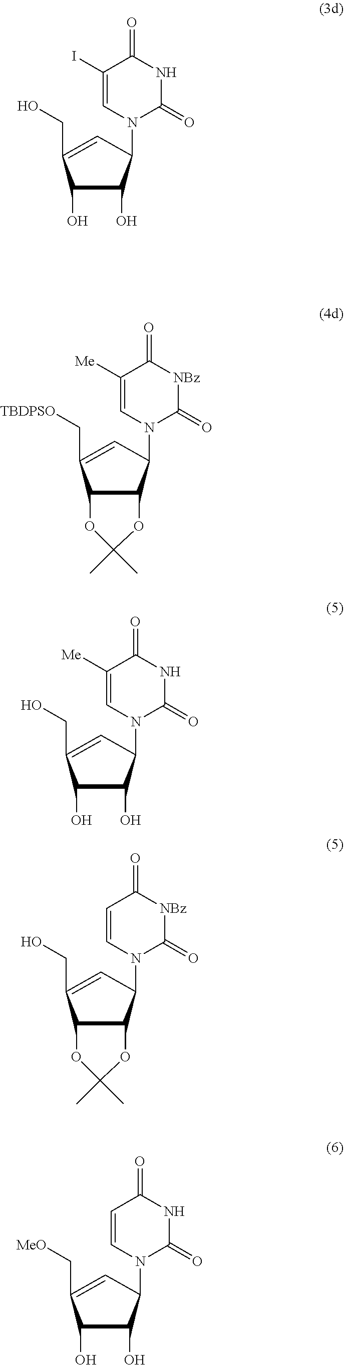

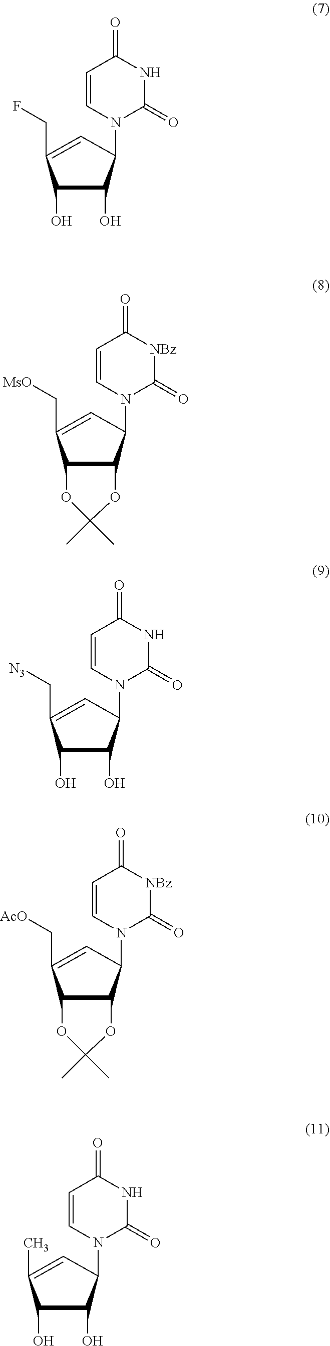

5. The method of claim 1, wherein the CPU analog is one or both of ((3aS,4R,6aR)-4-(3-benzoyl-2,4-dioxo-3,4-dihydropyrimidin-1(2H)-yl)-2,2-dimethyl-3a,6a-dihydro-4H-cyclopenta[d][1,3]dioxol-6-yl)methyl methanesulfonate, and 1-((1R,4R,5S)-4,5-dihydroxy-3-(hydroxymethyl)cyclopent-2-en-1-yl)-5-fluoropyrimidine-2,4(1H,3H)-dione.

6. The method of claim 1, wherein the CPU analog is selected from the group:
\textcircled{B} has a structure selected from the following group: 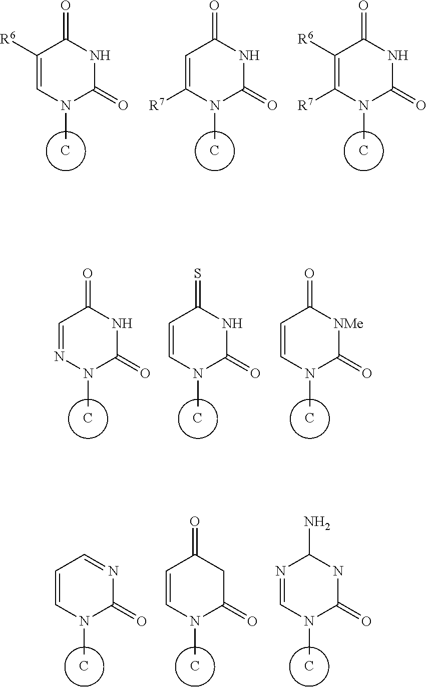
\textcircled{C} has a structure selected from the following group: 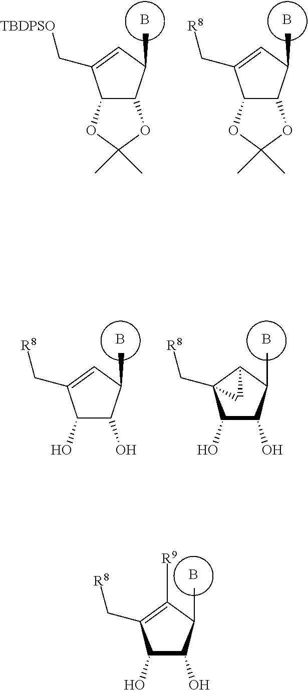
R⁶, R⁷, R^{,} and R⁹ is the same or different and is independently selected from the group consisting of halogen, haloalkyl, alkyl, alkenyl, alkynyl, cycloalkyl, cycloalkenyl, —R¹⁰cycloalkyl, Ay, —NHR¹⁰Ay, Het, —NHHet, —NHR¹⁰Het, —OR², —OAy, —OHet, —R¹⁰OR², —NR²R³, —NR²Ay, —R¹⁰NR²R³, —R¹⁰NR²Ay, —R¹⁰C(O)R², —C(O)R², —CO₂R², —R¹⁰CO₂R², —C(O)NR²R³, —C(O)Ay, —C(O)NR²Ay, —C(O)Het, —C(O)NHR¹⁰Het, —R¹⁰C(O)NR²R³, —C(S)NR²R³, —R¹⁰C(S)NR²R³, —R¹⁰NHC(NH)NR²R³, —C(NH)NR²R³, —R¹⁰C(NH)NR²R³, —S(O)₂NR²R³, —S(O)₂NR²Ay, —R¹⁰SO₂NHCOR², —R¹⁰SO₂NR²R³, —R¹⁰SO₂R², —S(O)ₘR², —S(O)ₘAy, cyano, nitro, or azido;
each m independently is 0, 1, or 2;
each R¹⁰ is the same or different and is independently selected from alkylene, cycloalkylene, alkenylene, cycloalkenylene, and alkynylene;
each of R² and R³ are the same or different and are independently selected from the group consisting of H, alkyl, alkenyl, alkynyl, cycloalkyl, cycloalkenyl, —R¹⁰cycloalkyl, —R¹⁰OH, —R¹⁰(OR¹⁰)_{w}, and —R¹⁰NR⁴R⁵;
w is 1-10;
each of R⁴ and R⁵ are the same or different and are independently selected from the group consisting of alkyl, cycloalkyl, alkenyl, cycloalkenyl, and alkynyl;
Ay represents an aryl group; Het represent a 5- or 6-membered heterocyclyl or heteroaryl group; ring A is aryl or heteroaryl; provided that when the A ring is aryl, t is 0, and Y is SO₂, then p is not 0;
including salt, solvates and physiologically functional derivatives thereof.

7. The method of claim 1, wherein the DHODH inhibitor has a structure of Formula I.

8. The method of claim 1, wherein the DHODH inhibitor is GSK983 or an analog thereof.

9. The method of claim 1, wherein the DHODH inhibitor is selected from leflunomide, teriflunomide, and brequinar.

10. The method of claim 3, wherein the RdRp inhibitor is a non-nucleotide/nucleoside inhibitor.

11. The method of claim 3, wherein the RdRp inhibitor is a nucleotide/nucleoside analog.

12. The method of claim 11, wherein the RdRp inhibitor is a cytidine analog.

13. The method of claim 12, wherein the RdRp inhibitor is 4-azidocytidine or its prodrug balapiravir.

14. The method of claim 3, wherein the RdRp inhibitor is selected from Favipiravir (T-705); NSC-320218; pyridoxal-5′-phosphate-6-(2′-naphthylazo-6′-nitro-4′,8′-disulfonate) tetrasodium salt (PPNDS); Celgosivir, NITD-008, NITD107, Balapiravir; functionalized 2,1-benzothiazine 2,2-dioxide; 5(1H)-Quinazolinone,2-(4-bromophenyl)-2,3,4,6,7,8-hexahydro-7,7-dimethyl-1,3-diphenyl (Q63); Sofosbuvir, Daclatasvir; 2-(3-Thienyl)-5,6-dihydroxypyrimidine-4-carboxylic acid; IDX375; R1479 (4′-azidocytidine); DMB213; Setrobuvir, YAK; IDX-184; 2-oxo-pyrazine-3-carboxamide-yl nucleoside analogues; 4-[(1S,3R,4R,7R)-7-hydroxy-1-(hydroxymethyl)-2,5-dioxabicyclo[2.2.1]heptan-3-yl]-3-oxo-3,4-dihydropyrazine-2-carboxamide; PC786; ALS-8112 and ALS-8176.

15. The method of claim 3, wherein the combined therapy provides for reduced toxicity toward host cells relative to administration of the RdRp inhibitor as a single agent.

16. The method of claim 3, wherein the combined therapy provides for improved therapeutic index of the RdRp inhibitor, relative to the therapeutic index of the RdRp inhibitor as a single agent.

17. The method of claim 1, wherein the subject is a human infected or exposed to the virus.

18. (canceled)

19. The method of claim 1, wherein the RNA virus is a dsRNA virus or a ssRNA virus.

20. (canceled)

21. (canceled)

22. (canceled)

23. (canceled)

24. A formulation, comprising:
(i) an effective dose of an inhibitor of dihydroorotate dehydrogenase (DHODH); and
(ii) an effective dose of a cyclopentenyl uracil (CPU) analog,
where the combined dose is effective to inhibit replication of a virus in a cell.

25. A formulation, comprising:
(i) an effective dose of an inhibitor of dihydroorotate dehydrogenase (DHODH); and
(ii) an effective dose of a cyclopentenyl uracil (CPU) analog, and
(ii) an effective dose of an inhibitor of RdRp.
where the combined dose is effective to inhibit replication of a virus in a cell.

26. (canceled)

27. (canceled)
